(19) United States
(12) Patent Application Publication
(10) Pub. No.: US 2022/0296657 A1
(43) Pub. Date: Sep. 22, 2022

(54) PREVOTELLA PREPARATIONS AND TREATING CHRONIC OBSTRUCTIVE PULMONARY DISEASE (COPD) AND OTHER LUNG CONDITIONS

(71) Applicant: Mayo Foundation for Medical Education and Research, Rochester, MN (US)

(72) Inventor: Veena Taneja, Rochester, MN (US)

(21) Appl. No.: 17/619,025

(22) PCT Filed: Jun. 17, 2020
(86) PCT No.: PCT/US2020/038084

§ 371 Date: Dec. 14, 2021

Related U.S. Application Data
(60) Provisional application No. 62/862,186, filed on Jun. 17, 2019.

(51) Int. Cl. A61K 35/741; A61K 9/00; A61P 11/00

(52) U.S. Cl. CPC A61K 35/741; A61K 9/0053; A61P 11/00

(57) ABSTRACT

This document provides methods and materials related to the treatment of a lung condition, such as chronic obstructive pulmonary disease (COPD), using a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof. For example, methods and materials for using a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof to treat a lung condition, such as COPD, are provided.

### CROSS-REFERENCE TO RELATED APPLICATIONS

This application claims priority to U.S. Provisional Application Ser. No. 62/862,186, filed on Jun. 17, 2019, incorporated by reference herein in its entirety.

### STATEMENT AS TO FEDERALLY SPONSORED RESEARCH

This invention was made with government support under AR060077 awarded by the National Institutes of Health. The government has certain rights in the invention.

### BACKGROUND

### 1. Technical Field

This document relates to Prevotella (e.g., P. histicola) preparations and the use of Prevotella (e.g., P. histicola) preparations to treat chronic obstructive pulmonary disease (COPD) and other lung conditions. This document also relates to (a) preparations of P. melaninogenica and (b) vesicles of a Prevotella species (e.g., P. histicola and/or P. melaninogenica) and the use of such preparations and/or such vesicles to treat a lung condition such as COPD.

### 2. Background Information

A large reservoir of microorganisms lives in the digestive tracts of animals and is often referred to as the gut flora or microflora. Bacteria make up most of the flora in the colon and about 60 percent of the dry mass of feces. In fact, between 300 and 1000 different species may live in the gut.

### SUMMARY

This document provides methods and materials related to treating COPD using a composition containing live and/or killed Prevotella (e.g., P. histicola). This document also provides compositions containing live and/or killed Prevotella (e.g., P. histicola) as well as methods and materials for making such compositions. For example, this document provides compositions containing live and/or killed Prevotella (e.g., P. histicola) in the form of an oral medicament or dietary supplement (e.g., a pill, tablet, capsule). In some cases, a composition containing live and/or killed Prevotella (e.g., P. histicola) provided herein can be used as an oral COPD medicament or dietary supplement to treat COPD. In some cases, a composition containing live and/or killed Prevotella (e.g., P. histicola) provided herein can be used as a respiratory system (e.g., nasal) COPD medicament to treat COPD.

The compositions containing live and/or killed Prevotella (e.g., P. histicola) provided herein and the methods for using such compositions as described herein can allow medical professionals to treat subjects (e.g., mammal (e.g., human) patients) suffering from COPD effectively. In some cases, the methods and materials provided herein can allow humans to supplement their diets with bacterial organisms having the ability to reduce the severity or development of COPD.

In general, one aspect of this document features a method for treating chronic obstructive pulmonary lung disease (COPD) in a mammal. The method comprises (or consists essentially of or consists of) administering a composition comprising live or killed Prevotella to the mammal. The composition can comprise live Prevotella. The composition can comprise killed Prevotella and no live Prevotella. The mammal can be a human. The mammal can be a rodent. Administering can comprise oral administration. The composition can be a pill, tablet, or capsule. The composition can be a pill, tablet, or capsule configured to deliver the Prevotella to the intestines of the mammal. Administering can comprise administration to the respiratory system. The composition can be administered using a nasal spray, an inhaler, or a nebulizer. The severity of a symptom of the COPD can be reduced following the administering. The severity of the symptom of the COPD can be reduced by greater than about 25 percent following the administering. The severity of the symptom of the COPD can be reduced by greater than about 50 percent following the administering. The severity of the symptom of the COPD can be reduced by greater than about 75 percent following the administering. Lung compliance of the mammal can be decreased following the administering step. Lung compliance after the administering is decreased by at least about 5% as compared to before the administering. Lung compliance after the administering can be decreased by at least about 10% as compared to before the administering. Lung compliance after the administering can be decreased by at least about 15% as compared to before the administering. The method can further comprise measuring one or more parameters of lung function before the administering, and measuring the same one or more parameters of lung function following the administering. At least one of the one or more parameters of lung function measured following the administering can be improved compared to before the administering. The one or more parameters of lung function can comprise lung compliance, lung volume, airway resistance, elastance, or a combination thereof. The method can comprise identifying the mammal as having COPD prior to the administering step. The method can comprise identifying the mammal as having emphysema prior to the administering step. Representative cells of the Prevotella histicola can be those that are deposited as NRRL accession number B-50329. The composition can comprise about 1×10³ to about 1×10¹⁴ CFU of Prevotella. The composition can comprise about 1×10⁵ to about 1×10¹² CFU of Prevotella. The composition can comprise about 1×10⁸ to about 1×10¹⁰ CFU of Prevotella. The composition can be administered once per day. The composition can be administered twice per day. The composition can be administered three times per day. The composition can be administered for a period of at least about 4 weeks. The composition can be administered for a period of at least about 8 weeks. The Prevotella can comprise (or consist essentially of or consist of) Prevotella histicola.

In another aspect, this document features a non-human mammalian model of chronic obstructive pulmonary disease (COPD). The somatic cells of the non-human mammalian model comprise nucleic acid encoding one or more human HLA serotypes, and wherein the somatic cells of the non-human mammalian model lack expression of endogenous MHC class II molecules and endogenous IL-17 polypeptides, and wherein, when exposed to cigarette smoke for about 2 to about 6 weeks, the model has a decrease in lung function as compared to a comparable model not exposed to cigarette smoke. The human HLA serotype can be DQ8. The model can be an IL-17^{-/-} knock-out mammal. The mammal can be a rodent. The mammal can be a mouse. The mammal can have a blood nicotine level of about 45 to about 185 ng/mL. The mammal can have a blood nicotine level of about 50 to about 150 ng/mL. The mammal can have an increased level of expression in CXCR3, CXCL10, or both, as compared to a comparable mammal that is not a non-human animal model of COPD. The mammal can have a decreased level of expression in one or more fibrosis-associated genes, as compared to a comparable mammal that is not a non-human animal model of COPD. At least one of the fibrosis-associated genes can be selected from the group consisting of collagen a1, fibronectin, and transforming growth factor beta. The decrease in lung function can be measured using one or more parameters of lung function. The one or more parameters of lung function can comprise lung compliance, lung volume, airway resistance, elastance, or a combination thereof.

In another aspect, this document features a method of producing a non-human mammalian model for chronic obstructive pulmonary disease (COPD). The method comprising (a) providing non-human mammal, wherein the somatic cells of the non-human mammalian comprise nucleic acid encoding one or more human HLA serotypes, and wherein the somatic cell of the non-human mammalian model lack expression of endogenous MHC class II molecules and endogenous IL-17 polypeptide; and (b) exposing the mammal to cigarette smoke for about 3 to about 6 weeks, thereby producing a non-human animal model for COPD. The human HLA serotype can be DQ8. The mammal can be IL-17^{-/-}. The mammal can be a rodent. The mammal can be a mouse. The exposing can comprise exposing the mammal to about 2 cigarettes about 10 minutes for about 3 hours per day about 5 days per week. The exposing can comprise sufficient exposure to cigarette smoke to result in a blood nicotine level of about 45 to about 185 ng/mL. The exposing can comprise sufficient exposure to cigarette smoke to result in a blood nicotine level of about 50 to about 150 ng/mL. Following the exposing, the mammal can have an increased level of expression in CXCR3, CXCL10, or both, as compared to a similar mammal exposed to air instead of cigarette smoke. Following the exposing, the mammal can have a decreased level of expression in one or more fibrosis-associated genes, as compared to a similar mammal exposed to air instead of cigarette smoke. At least one of the fibrosis-associated genes can be selected from the group consisting of collagen a1, fibronectin, and transforming growth factor beta. Following the exposing, the mammal can have a decrease in lung function as compared to a similar mammal exposed to air instead of cigarette smoke. The decrease in lung function can be measured using one or more parameters of lung function. The one or more parameters of lung function can comprise lung compliance, lung volume, airway resistance, elastance, or a combination thereof.

In another aspect, this document features a non-human mammalian model of COPD produced by a method comprising (a) providing non-human mammal, wherein the somatic cells of the non-human mammalian comprise nucleic acid encoding one or more human HLA serotypes, and wherein the somatic cell of the non-human mammalian model lack expression of endogenous MHC class II molecules and endogenous IL-17 polypeptide; and (b) exposing the mammal to cigarette smoke for about 3 to about 6 weeks, thereby producing a non-human animal model for COPD. The human HLA serotype can be DQ8. The mammal can be IL-17^{-/-}. The mammal can be a rodent. The mammal can be a mouse. The exposing can comprise exposing the mammal to about 2 cigarettes about 10 minutes for about 3 hours per day about 5 days per week. The exposing can comprise sufficient exposure to cigarette smoke to result in a blood nicotine level of about 45 to about 185 ng/mL. The exposing can comprise sufficient exposure to cigarette smoke to result in a blood nicotine level of about 50 to about 150 ng/mL. Following the exposing, the mammal can have an increased level of expression in CXCR3, CXCL10, or both, as compared to a similar mammal exposed to air instead of cigarette smoke. Following the exposing, the mammal can have a decreased level of expression in one or more fibrosis-associated genes, as compared to a similar mammal exposed to air instead of cigarette smoke. At least one of the fibrosis-associated genes can be selected from the group consisting of collagen a1, fibronectin, and transforming growth factor beta. Following the exposing, the mammal can have a decrease in lung function as compared to a similar mammal exposed to air instead of cigarette smoke. The decrease in lung function can be measured using one or more parameters of lung function. The one or more parameters of lung function can comprise lung compliance, lung volume, airway resistance, elastance, or a combination thereof.

In another aspect, this document features a method for treating a lung condition in a mammal. The method comprises (or consists essentially of or consists of) administering a composition comprising live or killed Prevotella to the mammal. The composition can comprise live Prevotella. The composition can comprise killed Prevotella and no live Prevotella. The mammal can be a human. The mammal can be a rodent. Administering can comprise oral administration. The composition can be a pill, tablet, or capsule. The composition can be a pill, tablet, or capsule configured to deliver the Prevotella to the intestines of the mammal. Administering can comprise administration to the respiratory system. The composition can be administered using a nasal spray, an inhaler, or a nebulizer. The severity of a symptom of the lung condition can be reduced following the administering. The severity of the symptom of the lung condition can be reduced by greater than about 25 percent following the administering. The severity of the symptom of the lung condition can be reduced by greater than about 50 percent following the administering. The severity of the symptom of the lung condition can be reduced by greater than about 75 percent following the administering. Lung compliance of the mammal can be decreased following the administering step. Lung compliance after the administering can be decreased by at least about 5% as compared to before the administering. Lung compliance after the administering can be decreased by at least about 10% as compared to before the administering. Lung compliance after the administering can be decreased by at least about 15% as compared to before the administering. The method can further comprise measuring one or more parameters of lung function before the administering, and measuring the same one or more parameters of lung function following the administering. At least one of the one or more parameters of lung function measured following the administering can be improved compared to before the administering. The one or more parameters of lung function comprise lung compliance, lung volume, airway resistance, elastance, or a combination thereof. The method can comprise identifying the mammal as having the lung condition prior to the administering step. The method can comprise identifying the mammal as having pneumonia prior to the administering step. Representative cells of the Prevotella can be deposited as NRRL accession number B-50329. The composition can comprise about 1×10³ to about 1×10¹⁴ CFU of Prevotella. The composition can comprise about 1×10⁵ to about 1×10¹² CFU of Prevotella. The composition can comprise about 1×10⁸ to about 1×10¹⁰ CFU of Prevotella. The composition can be administered once per day. The composition can be administered twice per day. The composition can be administered three times per day. The composition can be administered for a period of at least about 4 weeks. The composition can be administered for a period of at least about 8 weeks. The Prevotella can comprise Prevotella histicola. The lung condition can be selected from the group consisting of chronic obstructive pulmonary disease (COPD), asthma, pulmonary fibrosis, sarcoidosis, disease caused by infection, and combinations thereof. The disease caused by infection can be selected from the group consisting of a viral infection, a bacterial infection, a fungal infection, a parasitic infection, and combinations thereof. The viral infection can be selected from the group consisting of viral pneumonia, viral bronchitis, influenza, coronavirus infection, adenovirus infection, syncytial virus infection, rhinovirus infection, and combinations thereof. The bacterial infection can be selected from the group consisting of Streptococcus infection, Staphylococcus infection, Chlamydophila infection, Mycoplasma infection, Haemophilus infection, Corynebacterium infection, Mycobacterium infection, Legionella infection, and combinations thereof. The lung condition can comprise pneumonia.

In another aspect, this document features a method for treating a lung condition in a mammal. The method comprises (or consists essentially of or consists of) administering a composition comprising live or killed Prevotella melaninogenica to the mammal. The composition can comprise live Prevotella melaninogenica. The composition can comprise killed Prevotella melaninogenica and no live Prevotella melaninogenica. The mammal can be a human. The mammal can be a rodent. Administering can comprise oral administration. The composition can be a pill, tablet, or capsule. The composition can be a pill, tablet, or capsule configured to deliver the Prevotella melaninogenica to the intestines of the mammal. Administering can comprise administration to the respiratory system. The composition can be administered using a nasal spray, an inhaler, or a nebulizer. The severity of a symptom of the lung condition can be reduced following the administering. The severity of the symptom of the lung condition can be reduced by greater than about 25 percent following the administering. The severity of the symptom of the lung condition can be reduced by greater than about 50 percent following the administering. The severity of the symptom of the lung condition can be reduced by greater than about 75 percent following the administering. Lung compliance of the mammal can be decreased following the administering step. Lung compliance after the administering can be decreased by at least about 5% as compared to before the administering. Lung compliance after the administering can be decreased by at least about 10% as compared to before the administering. Lung compliance after the administering can be decreased by at least about 15% as compared to before the administering. The method can further comprise measuring one or more parameters of lung function before the administering, and measuring the same one or more parameters of lung function following the administering. At least one of the one or more parameters of lung function measured following the administering can be improved compared to before the administering. The one or more parameters of lung function comprise lung compliance, lung volume, airway resistance, elastance, or a combination thereof. The method can comprise identifying the mammal as having the lung condition prior to the administering step. The method can comprise identifying the mammal as having pneumonia prior to the administering step. The method can comprise identifying the mammal as having COPD prior to the administering step. The composition can comprise about 1×10³ to about 1×10¹⁴ CFU of Prevotella melaninogenica. The composition can comprise about 1×10⁵ to about 1×10¹² CFU of Prevotella melaninogenica. The composition can comprise about 1×10⁸ to about 1×10¹⁰ CFU of Prevotella melaninogenica. The composition can be administered once per day. The composition can be administered twice per day. The composition can be administered three times per day. The composition can be administered for a period of at least about 4 weeks. The composition can be administered for a period of at least about 6 weeks. The composition can be administered for a period of at least about 8 weeks. The lung condition can be selected from the group consisting of chronic obstructive pulmonary disease (COPD), asthma, pulmonary fibrosis, sarcoidosis, disease caused by infection, and combinations thereof. The disease caused by infection can be selected from the group consisting of a viral infection, a bacterial infection, a fungal infection, a parasitic infection, and combinations thereof. The viral infection can be selected from the group consisting of viral pneumonia, viral bronchitis, influenza, coronavirus infection, adenovirus infection, syncytial virus infection, rhinovirus infection, and combinations thereof. The bacterial infection can be selected from the group consisting of Streptococcus infection, Staphylococcus infection, Chlamydophila infection, Mycoplasma infection, Haemophilus infection, Corynebacterium infection, Mycobacterium infection, Legionella infection, and combinations thereof. The lung condition can comprise pneumonia.

In another aspect, this document features a method for treating a lung condition in a mammal. The method comprises (or consists essentially of or consists of) administering a composition comprising vesicles of a Prevotella species. The mammal can be a human. The mammal can be a rodent. Administering can comprise oral administration. The composition can be a pill, tablet, or capsule. The composition can be a pill, tablet, or capsule configured to deliver the vesicles of the Prevotella species to the intestines of the mammal. Administering can comprise administration to the respiratory system. The composition can be administered using a nasal spray, an inhaler, or a nebulizer. The severity of a symptom of the lung condition can be reduced following the administering. The severity of the symptom of the lung condition can be reduced by greater than about 25 percent following the administering. The severity of the symptom of the lung condition can be reduced by greater than about 50 percent following the administering. The severity of the symptom of the lung condition can be reduced by greater than about 75 percent following the administering. Lung compliance of the mammal can be decreased following the administering step. Lung compliance after the administering can be decreased by at least about 5% as compared to before the administering. Lung compliance after the administering can be decreased by at least about 10% as compared to before the administering. Lung compliance after the administering can be decreased by at least about 15% as compared to before the administering. The method can further comprise measuring one or more parameters of lung function before the administering, and measuring the same one or more parameters of lung function following the administering. At least one of the one or more parameters of lung function measured following the administering can be improved compared to before the administering. The one or more parameters of lung function comprise lung compliance, lung volume, airway resistance, elastance, or a combination thereof. The method can comprise identifying the mammal as having the lung condition prior to the administering step. The method can comprise identifying the mammal as having pneumonia prior to the administering step. Representative cells of the Prevotella species can be deposited as NRRL accession number B-50329. The composition can be administered once per day. The composition can be administered twice per day. The composition can be administered three times per day. The composition can be administered for a period of at least about 4 weeks. The composition can be administered for a period of at least about 8 weeks. The Prevotella species can comprise Prevotella histicola. The Prevotella species can comprise Prevotella melaninogenica. The lung condition can be selected from the group consisting of chronic obstructive pulmonary disease (COPD), asthma, pulmonary fibrosis, sarcoidosis, disease caused by infection, and combinations thereof. The disease caused by infection can be selected from the group consisting of a viral infection, a bacterial infection, a fungal infection, a parasitic infection, and combinations thereof. The viral infection can be selected from the group consisting of viral pneumonia, viral bronchitis, influenza, coronavirus infection, adenovirus infection, syncytial virus infection, rhinovirus infection, and combinations thereof. The bacterial infection can be selected from the group consisting of Streptococcus infection, Staphylococcus infection, Chlamydophila infection, Mycoplasma infection, Haemophilus infection, Corynebacterium infection, Mycobacterium infection, Legionella infection, and combinations thereof. The lung condition can comprise pneumonia.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a bar graph plotting expression of various proteins in lungs as measured by rtPCR in DQ8 mice exposed to cigarette smoke (CS) or air (NS) for 5 weeks. (N=3 mice/group)

FIG. 2A is a plot showing lung compliance (Cst) (as a PV curve) after exposure to air (NS) for 4 weeks in DQ8 mice. N=7.

FIG. 2B is a plot showing lung compliance (Cst) (as a PV curve) after exposure to CS for 4 weeks in DQ8 mice. N=7.

FIG. 2C is a plot showing lung compliance (Cst) (as a PV curve) after exposure to CS for 4 weeks in DQ8.IL-17^{-/-} mice. N=4.

FIG. 3 is a bar graph plotting compiled data from each group in FIGS. 2A-2C.

FIG. 4 shows exemplary P. histicola 16S rRNA sequences (SEQ ID NO:1 and SEQ ID NO: 2).

FIG. 5A shows representative PV curves of lung compliance for CS exposed DQ8 mice.

FIG. 5B shows representative PV curves of lung compliance for CS exposed DQ8 mice treated with P. histicola.

FIG. 5C shows representative PV curves of lung compliance for CS exposed DQ8 mice treated with P. melaninogenica.

FIG. 5D is a plot of the lung compliance of naïve DQ8 mice, and the mice from FIGS. 5A-5C.

FIG. 6 is a plot of the lung compliance of naïve DQ8.IL17−/− mice (N), and the same type of mice treated with cigarette smoke (CS), treated with cigarette smoke and P. histicola (CS/PH), or treated with cigarette smoke and P. melaninogenica (CS/PM).

FIG. 7 is a plot of the lung compliance of DQ8 mice treated with CS (CS), and the same type of mice treated with outer membrane vesicles (OMVs) of P. histicola (CS/PH/OMV) or with OMVs of P. melaninogencia (CS/PM/OMV).

FIG. 8A is a plot of IL-1b transcription in DQ8 mice treated with CS (DQ8 CS Naïve) or treated with CS and P. histicola (DQ8 Naïve CS P. hist).

FIG. 8B is a plot of TNF transcription in DQ8 mice treated with CS (DQ8 CS Naïve) or treated with CS and P. histicola (DQ8 Naïve CS P. hist).

FIG. 8C is a plot of IL-23a transcription in DQ8 mice treated with CS (DQ8 CS Naïve) or treated with CS and P. histicola (DQ8 Naïve CS P. hist).

FIG. 8D is a plot of T CTLA-4 transcription in DQ8 mice treated with CS (DQ8 CS Naïve) or treated with CS and P. histicola (DQ8 Naïve CS P. hist).

FIG. 8E is a plot of IL-13 transcription in DQ8 mice treated with CS (DQ8 CS Naïve) or treated with CS and P. histicola (DQ8 Naïve CS P. hist).

FIG. 9A is a plot of IL-1b transcription in DQ8 mice: naïve mice (Naïve), CS-treated mice (DQ8 CS Naïve), and mice treated with both CS and P. histicola (DQ8 Naïve CS P. hist).

FIG. 9B is a plot of T CTLA-4 transcription in DQ8 mice: naïve mice (Naïve), CS-treated mice (DQ8 CS Naïve), and mice treated with both CS and P. histicola (DQ8 Naïve CS P. hist).

FIG. 10A is a plot of IL-17R transcription in DQ8 mice treated with CS (DQ8 CS Naïve) or treated with CS and P. histicola (DQ8 Naïve CS P. hist).

FIG. 10B is a plot of IL-6 transcription in DQ8 mice treated with CS (DQ8 CS Naïve) or treated with CS and P. histicola (DQ8 Naïve CS P. hist).

FIG. 11 is a bar plot of lung compliance in mice exposed to CS for 2 weeks and immunized with type II collagen (CII), half of which were treated with Porphorymonas gingivalis.

### DETAILED DESCRIPTION

COPD is the fifth leading cause of death. It is believed that cigarette smoking is an important etiologic factor leading to the development of emphysema/COPD. Cigarette smoke (CS) is believed to be a contributory factor in the pathogenesis of emphysema/COPD. COPD is sometimes characterized by an inflammation of the airways and emphysema that progresses to irreversible airflow limitation. Smoking cessation does not always impact disease progression, suggesting the involvement of endogenous factors besides smoking in the progression of disease. Given the limitations of current therapies to stop the progression of COPD, it is helpful to identify factors that are involved in pathogenesis for use as a target for treatment.

Cigarette smoking is a major environmental factor for enhancing autoimmunity and inflammatory diseases. The mechanism by which CS causes inflammation has been studied extensively. It is believed that CS promotes immune dysregulation by inducing influx of neutrophils and other pro-inflammatory cells that secrete IL-17. Differentiation of Th17 cells by mucosa-associated commensals contributes to protection from infections. The lungs are not sterile, and lung microbiota may be a factor in CS-induced inflammation in the lung. This concept is supported by the role of gut microbiota in regulating adaptive and innate immune responses. There is limited information on the role of lung microbiome in COPD. The lungs of smokers and COPD lungs harbor alterations in microbiome composition as compared to healthy lungs (see, e.g., Yadava, K., et al. Am J Respir Crit Care Med 193: 975-987, 2016; Wang, Z., et al. Thorax 73: 331-338 (2018); Larsen, J. Immunology 151.4: 363-374 (2017); Scher, J., et al. Microbiome 4.1:60 (2016)). In some cases, a closer relation between lung and oral microbiota than lung and nasal microbiota has been observed (see, e.g., Bassis, C. M., et al. MBio 6: e00037 (2015)). The COPD microbiome can have an abundance of Pseudomonas and Haemophilus but can lack commonly present oral and gut microbes belonging to Prevotella genus (see, e.g., Wang, Z., et al. Thorax 73: 331-338 (2018)).

Any appropriate species, or combination of species, of Prevotella can be used for any of the methods described herein. In some embodiments, P. histicola can be used. In some embodiments, P. melaninogenica can be used. In some embodiments, P. histicola and P. melaninogenica can be used. In some cases, a combination of Prevotella species can balance immune response and improve lung function in a lung condition (e.g., any of the lung conditions described herein, for example, COPD).

This document provides methods for treating a lung condition in a mammal. The methods can include administering to the mammal a composition that includes (a) Prevotella (e.g., P. histicola and/or P. melaninogenica, and/or (b) vesicles of a Prevotella species (e.g., P. histicola and/or P. melaninogenica). Examples of lung conditions that can be treated as described herein include, without limitation, COPD, asthma, pulmonary fibrosis, sarcoidosis, and infections (e.g., viral, bacterial, parasitic, or fungal infections) that affect the lung or that affect lung function. In some embodiments, such an infection can be a viral infection (e.g., viral pneumonia, viral bronchitis, influenza, coronavirus infection, adenovirus infection, syncytial virus infection, rhinovirus infection, or a combination thereof), a bacterial infection (e.g., Streptococcus infection, Staphylococcus infection, Chlamydophila infection, Mycoplasma infection, Haemophilus infection, Corynebacterium infection, Mycobacterium infection, Legionella infection, or a combination thereof), a fungal infection (e.g., Aspergillus infection, Cryptococcus infection, Pneumocystis infection, or a combination thereof), a parasitic infection (e.g., Plasmodium infection, Toxoplasma infection, Entamoeba infection, Ascaris infection, Schistosoma infection, or a combination thereof), or a combination thereof. In some embodiments, a lung condition can be pneumonia.

This document also provides methods for treating COPD in a mammal. The methods can include administering to the mammal a composition containing (a) Prevotella (e.g., P. histicola and/or P. melaninogenica) and/or (b) vesicles of a Prevotella species (e.g., P. histicola and/or P. melaninogenica).

As used herein a “vesicle” of a Prevotella species can refer to any membrane vesicle produced by the Prevotella species. In some embodiments, a vesicle of a Prevotella species can be an outer membrane vesicle. Extracellular vesicles produced by the gram negative bacteria are typically called outer membrane vesicles (OMV). Without being bound by any particular theory, it is believed that because a whole microbe can be involved in other functions, use of vesicles (e.g., OMVs) from a Prevotella species may reduce or eliminate any potential pathogenic effects as compared to the intact, live bacteria.

In some embodiments, administering a composition containing Prevotella (e.g., P. histicola) can include administering a composition containing live Prevotella (e.g., P. histicola), dead (or killed) Prevotella (e.g., P. histicola), components of Prevotella (e.g., P. histicola), or a combination thereof. In some cases, administering a composition containing Prevotella (e.g., P. histicola and/or P. melaninogenica) can include administering a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), or a combination thereof.

As used herein, a “component” of a bacterium can be a part or a piece of a bacterium that is not a vesicle. In some embodiments, a component of a bacterium can be a fragment of a cell wall and/or membrane. In some embodiments, a component of a bacterium can be a protein.

In some embodiments, a mammal can be identified as having one or more symptoms of a lung condition (e.g., COPD) prior to administering a composition containing (a) live and/or killed Prevotella (e.g., P. histicola and/or P. melaninogenica) and/or (b) vesicles of a Prevotella species (e.g., P. histicola and/or P. melaninogenica). Examples of symptoms of a lung condition can vary based on the condition, but can include, without limitation, shortness of breath, wheezing, chest tightness and/or pain, cough, fever, and hoarseness. In some cases, a mammal can have a history of smoking or exposure to cigarette smoke. In some embodiments, a mammal can be identified or diagnosed as having a lung condition prior to administering a composition containing (a) live and/or killed Prevotella (e.g., P. histicola and/or P. melaninogenica) and/or (b) vesicles of a Prevotella species (e.g., P. histicola and/or P. melaninogenica).

In some embodiments, a mammal can be identified as having one or more symptoms of COPD prior to administering a composition containing live and/or killed Prevotella (e.g., P. histicola). In some embodiments, a mammal can be identified as having one or more symptoms of COPD prior to administering a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof.

Examples of symptoms of COPD include, without limitation, shortness of breath, wheezing, chest tightness, chronic cough, and cyanosis. In some cases, a mammal can have a history of smoking or exposure to cigarette smoke. In some embodiments, a mammal can be identified or diagnosed as having COPD prior to administering a composition containing live and/or killed Prevotella (e.g., P. histicola). In some embodiments, a mammal can be identified or diagnosed as having COPD prior to administering a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof.

In some embodiments, a mammal can be identified or diagnosed as having emphysema prior to administering a composition containing live and/or killed Prevotella (e.g., P. histicola).

In some embodiments, a mammal can be identified or diagnosed as having emphysema prior to administering a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof. In some embodiments, a mammal having COPD and emphysema can be treating using a method that includes administering a composition containing live and/or killed Prevotella (e.g., P. histicola) to the mammal. In some embodiments, a mammal having COPD and emphysema can be treating using a method that includes administering a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof to the mammal.

In some embodiments, the Prevotella can include Prevotella histicola. In some embodiments, the Prevotella can be predominantly Prevotella histicola. In some embodiments, the Prevotella can be nearly all Prevotella histicola. In some embodiments, the Prevotella can be all Prevotella histicola. In some cases, greater than 80 percent (e.g., greater than 85, 90, 95, or 99 percent) of the Prevotella of a composition to be administered to a mammal as described herein can be Prevotella histicola.

In some embodiments, the Prevotella can include Prevotella melaninogenica. In some embodiments, the Prevotella can be predominantly Prevotella melaninogenica. In some embodiments, the Prevotella can be nearly all Prevotella melaninogenica. In some embodiments, the Prevotella can be all Prevotella melaninogenica. In some cases, greater than 80 percent (e.g., greater than 85, 90, 95, or 99 percent) of the Prevotella of a composition to be administered to a mammal as described herein can be Prevotella melaninogenica. In some embodiments, vesicles of the Prevotella species can include vesicles of Prevotella histicola. In some embodiments, vesicles of the Prevotella species can predominantly include vesicles of Prevotella histicola. In some embodiments, vesicles of the Prevotella species can be nearly all vesicles of Prevotella histicola. In some embodiments, vesicles of the Prevotella species can be all vesicles of Prevotella histicola. In some cases, greater than 80 percent (e.g., greater than 85, 90, 95, or 99 percent) of the vesicles of Prevotella of a composition to be administered to a mammal as described herein can be vesicles of Prevotella histicola.

In some embodiments, vesicles of the Prevotella species can include vesicles of Prevotella melaninogenica. In some embodiments, vesicles of the Prevotella species can predominantly include vesicles of Prevotella melaninogenica. In some embodiments, vesicles of the Prevotella species can be nearly all vesicles of Prevotella melaninogenica. In some embodiments, vesicles of the Prevotella species can be all vesicles of Prevotella melaninogenica. In some cases, greater than 80 percent (e.g., greater than 85, 90, 95, or 99 percent) of the vesicles of Prevotella of a composition to be administered to a mammal as described herein can be vesicles of Prevotella melaninogenica.

In some embodiments, vesicles of the Prevotella species can include vesicles of Prevotella histicola and vesicles of Prevotella melaninogenica, in any appropriate ratio (e.g., 70:30, 60:40, 50:50, 40:60, or 30:70).

Some Prevotella bacteria are part of the human microbiome, though in some cases, some Prevotella bacteria can be pathogenic or opportunistic pathogens; see, e.g., de Steenhuijsen Piters et al., “Dysbiosis of upper respiratory tract microbiota in elderly pneumonia patients.” ISME J., 10:97-108 (2016). doi: 10.1038/ismej.2015.99.

Methods provided herein can be performed on any appropriate mammal. For example, in some embodiments, the mammal can be a human. Examples of mammals having a lung condition that can be treated as described herein include, without limitation, humans, monkeys, horses, bovine species, pigs, dogs, cats, goats, sheep, rabbits, guinea pigs, rats, and mice. Examples of mammals having COPD and/or emphysema that can be treated as described herein include, without limitation, humans, monkeys, horses, bovine species, pigs, dogs, cats, goats, sheep, rabbits, guinea pigs, rats, and mice.

Administering a composition containing live and/or killed Prevotella (e.g., P. histicola) can be performed in any appropriate manner. For example, a composition provided herein containing Prevotella (e.g., P. histicola) (e.g., live Prevotella (e.g., P. histicola) microorganisms) can be administered as described elsewhere for probiotic bacteria (see, e.g., paragraphs [0049]-[0108] of U.S. Patent Application Publication No. 2008/0241226; and column 5, line 53 to column 8, line 8 of U.S. Pat. No. 8,617,536, both of which are incorporated herein by reference in their entirety).

In some embodiments, a composition containing Prevotella (e.g., P. histicola) described herein can be administered orally. In some embodiments, the composition containing Prevotella (e.g., P. histicola) as described herein can be administered in the form of a pill, tablet or capsule. In some embodiments, a pill, tablet, or capsule can be configured to deliver the Prevotella (e.g., P. histicola) to the intestines of the mammal.

In some cases, a composition containing Prevotella (e.g., P. histicola) described herein can be administered to the respiratory system of the mammal. In some embodiments, a composition containing Prevotella (e.g., P. histicola) described herein can be administered nasally. A composition containing Prevotella (e.g., P. histicola) as described herein can be administered to the respiratory system of the mammal using any appropriate device or method. For example, a composition containing Prevotella (e.g., P. histicola) described herein can be administered to the respiratory system of the mammal using a nasal spray, an inhaler (e.g., a metered-dose inhaler or a dry powder inhaler), or a nebulizer (e.g., a jet nebulizer, an ultrasonic nebulizer, or a vibrating mesh nebulizer). In some embodiments, a composition comprising Prevotella (e.g., P. histicola) as described herein can be administered intranasally.

Administering a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be performed in any appropriate manner. For example, a composition provided herein containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be administered as described elsewhere for probiotic bacteria (see, e.g., paragraphs [0049]-[0108] of U.S. Patent Application Publication No. 2008/0241226; and column 5, line 53 to column 8, line 8 of U.S. Pat. No. 8,617,536, both of which are incorporated herein by reference in their entirety).

In some embodiments, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof described herein can be administered orally. In some embodiments, the composition live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof as described herein can be administered in the form of a pill, tablet or capsule. In some embodiments, a pill, tablet, or capsule can be configured to deliver the live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof to the intestines of the mammal. In some embodiments, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof described herein can be administered intranasally.

In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof as described herein can be administered to the respiratory system of the mammal. In some embodiments, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof described herein can be administered nasally. A composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof as described herein can be administered to the respiratory system of the mammal using any appropriate device or method. For example, a composition live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof as described herein can be administered to the respiratory system of the mammal using a nasal spray, an inhaler (e.g., a metered-dose inhaler or a dry powder inhaler), or a nebulizer (e.g., a jet nebulizer, an ultrasonic nebulizer, or a vibrating mesh nebulizer).

Any appropriate dosing schedule can be used to treat a lung condition as described herein. Any appropriate dosing schedule can be used to treat COPD and/or emphysema as described herein. For example, a composition provided herein can be administered once a day, twice a day, three times a day, or four times a day, with or without food.

An administration of a composition described herein can occur for any appropriate period of time. For example, a composition provided herein can be administered for a period of 1 week to about 10 weeks (e.g., about 1 week to about 8 weeks, about 1 week to about 4 weeks, about 1 week to about 2 weeks, about 2 weeks to about 4 weeks, about 2 weeks to about 8 weeks, about 2 weeks to about 10 weeks, about 4 weeks to about 10 weeks, about 8 weeks to about 10 weeks, about 2 weeks to about 8 weeks, or about 3 weeks to about 5 weeks). As another example, a composition described herein can be administered for a period of at least about 2 weeks (e.g., at least about 4 weeks, at least about 8 weeks, at least about 12 weeks, at least about 18 weeks, or at least about 24 weeks). In some cases, a composition described herein can be administered for as long as the mammal is alive.

In some cases, a mammal receiving a composition described herein can be monitored to determine the effect of the composition on lung function (e.g., to confirm the reduction of one or more symptoms of a lung condition such as COPD). Before, during (e.g., between doses), or after cessation of administration, lung function can be evaluated using any appropriate method. For example, lung function can be evaluated by measuring lung compliance (CST; also called pulmonary compliance), lung volume, airway resistance, or elastance. Any appropriate method can be used to measure a lung function. In some cases, a measure of lung function can be compared between two time points (e.g., before administrating a composition described herein and after one or more weeks of administration; between one dose and a subsequent dose; or before administering and after cessation of administration) to determine whether there was a change in the measure of lung function. In some embodiments of the methods provided herein, the methods can include measuring one or more parameters of lung function before, during, or after administering a composition described herein (e.g., after administering a composition described herein for at least 1 week (e.g., at least 2, 4, 8, 12, 18, or 24 weeks). In some embodiments, the one or more parameters of lung function after administering a composition described herein (e.g., after administering a composition described herein for at least 1 week (e.g., at least 2, 4, 8, 12, 18, or 24 weeks) can be improved as compared to the same one or more parameters of lung function before administering a composition described herein. In some embodiments, lung compliance of the mammal (e.g., a human) can be decreased by at least about 5% (e.g., at least about 6%, 8%, 10%, or 15%) after administering a composition described herein (e.g., after administering a composition described herein for at least 1 week (e.g., at least 2, 4, 8, 12, 18, or 24 weeks)) as compared to before the administering a composition described herein. In some embodiments of the methods provided herein, the severity of the lung condition such as COPD can be reduced after administering a composition described herein. For example, in some embodiments, the severity of the emphysema can be reduced by greater than 25%, greater than 50%, or greater than 75% after administering a composition described herein. A reduction in the severity of emphysema can be determined by any appropriate method. For example, a reduction in the severity of emphysema can be determined by measuring lung compliance.

Any appropriate composition containing Prevotella (e.g., P. histicola) can be used as described herein. Any appropriate composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereofcan be used as described herein.

Examples of compositions containing Prevotella (e.g., P. histicola) that can be used as described herein to treat COPD and/or emphysema include, without limitation, those described in the section extending from column 5, line 53 to column 8, line 8 of U.S. Pat. No. 8,617,536, incorporated herein by reference in its entirety. Examples of compositions containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof) that can be used as described herein to treat COPD and/or emphysema include, without limitation, those described in the section extending from column 5, line 53 to column 8, line 8 of U.S. Pat. No. 8,617,536, incorporated herein by reference in its entirety.

Examples of compositions containing Prevotella (e.g., P. histicola) that can be used as described herein to treat a lung condition include, without limitation, those described in the section extending from column 5, line 53 to column 8, line 8 of U.S. Pat. No. 8,617,536, incorporated herein by reference in its entirety. Examples of compositions containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof) that can be used as described herein to treat a lung condition include, without limitation, those described in the section extending from column 5, line 53 to column 8, line 8 of U.S. Pat. No. 8,617,536, incorporated herein by reference in its entirety.

Briefly, compositions containing Prevotella can include live Prevotella (e.g., P. histicola and/or P. melaninogenica), killed (also called dead) Prevotella (e.g., P. histicola and/or P. melaninogenica), Prevotella (e.g., P. histicola and/or P. melaninogenica) components, lysed Prevotella (e.g., P. histicola and/or P. melaninogenica), vesicles (e.g., OMVs) of Prevotella (e.g., P. histicola and/or P. melaninogenica), or a combination thereof. Such compositions can contain any appropriate amount of Prevotella (e.g., P. histicola and/or P. melaninogenica), Prevotella (e.g., P. histicola and/or P. melaninogenica) components, or a combination thereof. In some cases, a composition provided herein can contain live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), or a combination thereof in an amount such that between 0.001 and 100 percent (e.g., between 1 and 95 percent, between 10 and 95 percent, between 25 and 95 percent, between 50 and 95 percent, between 20 and 80 percent, between 50 and 95 percent, between 60 and 95 percent, between 70 and 95 percent, between 80 and 95 percent, between 90 and 95 percent, between 95 and 99 percent, between 50 and 100 percent, between 60 and 100 percent, between 70 and 100 percent, between 80 and 100 percent, between 90 and 100 percent, or between 95 and 100 percent), by dry weight, of the composition can be live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), or a combination thereof. In some cases, a composition provided herein can contain between about 10 and about 10¹⁴ live Prevotella (e.g., P. histicola) microorganisms. In some cases, a composition provided herein can contain any appropriate amount of vesicles of a Prevotella species (e.g., P. histicola and/or P. melaninogenica).

In some cases, a composition provided herein can contain Prevotella (e.g., P. histicola), P. melaninogenica, or a combination thereof (e.g., live Prevotella (e.g., P. histicola), or P. melaninogenica, microorganisms, killed Prevotella (e.g., P. histicola), or P. melaninogenica, microorganisms, or a combination thereof) in the amounts and dosages as described elsewhere for probiotic bacteria (U.S. Patent Application Publication No. 2008/0241226, incorporated herein by reference in its entirety; see, e.g., paragraphs [0049-0103]). In some cases, a composition provided herein can contain live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof in the amounts and dosages as described elsewhere for probiotic bacteria (U.S. Patent Application Publication No. 2008/0241226, incorporated herein by reference in its entirety; see, e.g., paragraphs [0049-0103]). Any appropriate dosage can be used. For example, a dosage of live Prevotella (e.g., P. histicola) can be about from 1×10³ to about 1×10¹⁴ colony forming units (CFU) (e.g., about 1×10³ to about 1×10¹², about 1×10³ to about 1×10¹⁰, about 1×10³ to about 1×10³, about 1×10³ to about 1×10⁵, about 1×10⁵ to about 1×10¹⁴, about 10⁶ to about 10⁹, about 10⁷ to about 10⁸, about 1×10⁸ to about 1×10¹⁴, about 1×10¹⁰ to about 1×10¹⁴, about 1×10¹² to about 1×10¹⁴, about 1×10⁵ to about 1×10¹², or about 1×10⁸ to about 1×10¹⁰ CFU). In some cases, a dosage of live Prevotella (e.g., P. histicola and/or P. melaninogenica) can be from about 1×10³ to about 1×10¹⁴ CFU (e.g., about 1×10³ to about 1×10¹², about 1×10³ to about 1×10¹⁰, about 1×10³ to about 1×10³, about 1×10³ to about 1×10⁵, about 1×10⁵ to about 1×10¹⁴, about 10⁶ to about 10⁹, about 10⁷ to about 10⁸, about 1×10⁸ to about 1×10¹⁴, about 1×10¹⁰ to about 1×10¹⁴, about 1×10¹² to about 1×10¹⁴, about 1×10⁵ to about 1×10¹², or about 1×10⁸ to about 1×10¹⁰ CFU). It will be understood that a dosage of killed Prevotella (e.g., P. histicola and/or P. melaninogenica) can be equivalent to a dosage of live Prevotella (e.g., P. histicola and/or P. melaninogenica), despite the killed Prevotella (e.g., P. histicola and/or P. melaninogenica) being unable to form colonies.

A dosage of vesicles of a Prevotella species (e.g., P. histicola and/or P. melaninogenica) can be any appropriate dosage. For example, a dosage of vesicles of a Prevotella species can be from about 10 mg OMV LPS (lipopolysaccharide) to about 1000 mg OMV LPS (e.g., about 10 mg OMV LPS to about 50 mg OMV LPS, about 10 mg OMV LPS to about 100 mg OMV LPS, about 10 mg OMV LPS to about 500 mg OMV LPS, about 50 mg OMV LPS to about 1000 mg OMV LPS, about 100 mg OMV LPS to about 1000 mg OMV LPS, about 500 mg OMV LPS to about 1000 mg OMV LPS, about 50 mg OMV LPS to about 500 mg OMV LPS, or about 50 mg OMV LPS to about 250 mg OMV LPS).

In some cases, a dosage can vary over a course of treatment (e.g., one or more high loading dosages can be used, followed by one or more lower dosages). It will be appreciated that an appropriate dosage can be impacted by several factors, including the age, weight, or severity of symptoms of a mammal to be treated.

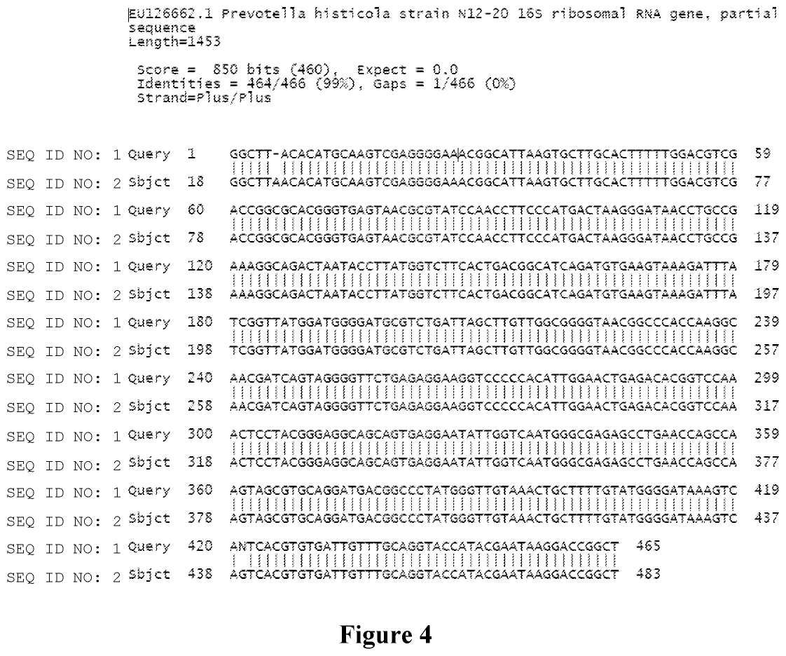

Live Prevotella (e.g., P. histicola and/or P. melaninogenica) microorganisms can be obtained from the digestive system of any appropriate mammal (e.g., a human). For example, Prevotella (e.g., P. histicola) microorganisms can be isolated from small intestinal mucosa (e.g., a small bowel biopsy or aspirate sample) of a human (e.g., a human patient diagnosed with celiac disease). Prevotella (e.g., P. histicola and/or P. melaninogenica) strains can be identified by any appropriate method, for example, via 16S rRNA PCR using standard 16S rRNA primers. A 16S rRNA sequence used to identify Prevotella (e.g., P. histicola) can be as set forth in FIG. 4 (e.g., SEQ ID NO: 1 and/or SEQ ID NO: 2). In some cases, Prevotella (e.g., P. histicola) microorganisms can be obtained from the ARS Culture Collection (NRRL accession number NRRL B-50329, deposited Oct. 28, 2009). Any appropriate method can be used to obtain a culture of Prevotella (e.g., P. histicola and/or P. melaninogenica) microorganisms. For example, standard microbial culturing techniques can be used to obtain Prevotella (e.g., P. histicola and/or P. melaninogenica) or Prevotella (e.g., P. histicola and/or P. melaninogenica) components. In general, Prevotella (e.g., P. histicola and/or P. melaninogenica) microorganisms can be cultured in broth containing milk (e.g., skim milk) to obtain a culture containing greater than 1×10⁸ Prevotella (e.g., P. histicola and/or P. melaninogenica) per mL of broth. The Prevotella (e.g., P. histicola and/or P. melaninogenica) microorganisms can be removed from the broth via centrifugation. Once obtained, the live Prevotella (e.g., P. histicola and/or P. melaninogenica) microorganisms can be formulated into a medicament or nutritional supplement composition for administration to a mammal (e.g., a human), can be added to a food product for consumption, or can be frozen for later use. In some cases, the obtained Prevotella (e.g., P. histicola and/or P. melaninogenica) microorganisms can be treated (e.g., chemical treatment, repeated freeze-thaw cycles, antibiotic treatment, a fixation treatment such a formalin treatment, irradiation, heat inactivation, and/or lyophilization) to obtain a composition of killed or lysed Prevotella (e.g., P. histicola and/or P. melaninogenica) microorganisms or can be processed (e.g., lysed followed by fractionation) to obtain a composition of Prevotella (e.g., P. histicola and/or P. melaninogenica) components.

In some cases, a Prevotella (e.g., P. histicola and/or P. melaninogenica) preparation, which can be stored frozen in 2× skim milk, can be thawed and grown on CDC Anaerobe Laked Sheep Blood Agar with kanamycin and vancomycin (KV) (Becton, Dickson and Company, Sparks, Md., product number 221846) in an anaerobe jar with AnaeroPack System (product number 10-01, Mitsubishi Gas Chemical America, Inc., New York, N.Y.). The culture can be incubated at 35-37° C. for at least 48 hours.

Vesicles (e.g., OMVs) of a Prevotella species (e.g., P. histicola and/or P. melaninogenica) can be obtained by any appropriate method. For example, in some cases, vesicles of a Prevotella species (e.g., P. histicola and/or P. melaninogenica) can be obtained by filtering (e.g., to remove bacteria) a culture supernatant of the Prevotella species (e.g., resulting from a lower-force centrifugation, such as 10 k×g), isolating the vesicles by centrifugation (e.g., using a higher-force centrifugation, such as 400 k×g), and optionally washing the vesicles.

A composition containing Prevotella (e.g., P. histicola) or Prevotella (e.g., P. histicola) components can, in some cases, be in the form of an oral medicament or nutritional supplement. For example, compositions containing Prevotella (e.g., P. histicola) or Prevotella (e.g., P. histicola) components can be in the form of a pill, tablet, powder, liquid, or capsule. Tablets or capsules can be prepared by any appropriate method with pharmaceutically acceptable excipients such as binding agents, fillers, lubricants, disintegrants, or wetting agents. The tablets can be coated by methods known in the art. A composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can, in some cases, be in the form of an oral medicament or nutritional supplement. For example, compositions containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be in the form of a pill, tablet, powder, liquid, or capsule.

In some cases, a composition containing Prevotella (e.g., P. histicola) or Prevotella (e.g., P. histicola) components can be formulated such that live or killed Prevotella (e.g., P. histicola) or Prevotella (e.g., P. histicola) components are encapsulated for release within the intestines of a mammal. In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be formulated such that live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof are encapsulated for release within the intestines of a mammal.

Liquid preparations for oral administration can take the form of, for example, solutions, syrups, or suspension, or they can be presented as a dry product for constitution with saline or other suitable liquid vehicle before use. In some cases, a composition provided herein containing Prevotella (e.g., P. histicola) (e.g., live Prevotella (e.g., P. histicola) microorganisms, killed Prevotella (e.g., P. histicola) microorganisms, or a combination thereof) can be in a dosage form as described elsewhere (U.S. Patent Application Publication No. 2008/0241226; see, e.g., paragraphs [0129-0135]). For example, a composition provided herein can be in the form of a food product formulated to contain Prevotella (e.g., P. histicola) (e.g., live Prevotella (e.g., P. histicola) microorganisms) or Prevotella (e.g., P. histicola) components. In some cases, a composition provided herein live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be in a dosage form as described elsewhere (U.S. Patent Application Publication No. 2008/0241226; see, e.g., paragraphs [0129-0135]). For example, a composition provided herein can be in the form of a food product formulated to contain live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof. Examples of such food products include, without limitation, milk (e.g., acidified milk), yogurt, milk powder, tea, juice, beverages, candies, chocolates, chewable bars, cookies, wafers, crackers, cereals, treats, and combinations thereof.

In some cases, a composition containing Prevotella (e.g., P. histicola) or Prevotella (e.g., P. histicola) components can, in some cases, be in the form of a medicament for administration to the respiratory system, for example, for nasal administration or for pulmonary administration. In some cases, a composition containing Prevotella (e.g., P. histicola) or Prevotella (e.g., P. histicola) components can be provided in the form of a nasal spray, an inhaler (e.g., a metered-dose inhaler or a dry powder inhaler), or a formulation for nebulization (e.g., for use in a jet nebulizer, an ultrasonic nebulizer, or a vibrating mesh nebulizer). In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can, in some cases, be in the form of a medicament for administration to the respiratory system, for example, for nasal administration or for pulmonary administration. In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be provided in the form of a nasal spray, an inhaler (e.g., a metered-dose inhaler or a dry powder inhaler), or a formulation for nebulization (e.g., for use in a jet nebulizer, an ultrasonic nebulizer, or a vibrating mesh nebulizer). These formulations can be formed by any appropriate method using pharmaceutically acceptable excipients. In some such formulations, Prevotella (e.g., P. histicola) or Prevotella (e.g., P. histicola) components can be present in any appropriate dosage. In some such formulations, live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be present in any appropriate dosage.

A composition containing Prevotella (e.g., P. histicola) or Prevotella (e.g., P. histicola) components can contain other ingredients such as buffers, radical scavengers, antioxidants, reducing agents, or mixtures thereof. For example, a composition containing live Prevotella (e.g., P. histicola) can be formulated to contain botanicals, vitamins, minerals, or combinations thereof. In some cases, a composition provided herein containing Prevotella (e.g., P. histicola) (e.g., live Prevotella (e.g., P. histicola) microorganisms, killed Prevotella (e.g., P. histicola) microorganisms, or a combination thereof) can contain other ingredients as described elsewhere (U.S. Patent Application Publication No. 2008/0241226; see, e.g., paragraphs [0104-0128]).

A composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can contain other ingredients such as buffers, radical scavengers, antioxidants, reducing agents, or mixtures thereof. For example, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be formulated to contain botanicals, vitamins, minerals, or combinations thereof. In some cases, a composition provided herein containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can contain other ingredients as described elsewhere (U.S. Patent Application Publication No. 2008/0241226; see, e.g., paragraphs [0104-0128]).

In some cases, a composition containing Prevotella (e.g., P. histicola) or Prevotella (e.g., P. histicola) components can contain a pharmaceutically acceptable carrier for administration to a mammal, including, without limitation, sterile aqueous or non-aqueous solutions, suspensions, and emulsions. In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can contain a pharmaceutically acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other known carriers for oral administration.

This document also provides methods and materials for using a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof as a medicament for a lung condition. In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be used as a nutritional supplement to supplement a mammal's diet with bacterial organisms having the ability to reduce the severity or development of a lung condition. Examples of mammals that can be treated as described herein include, without limitation, humans, monkeys, dogs, cats, cows, horses, pigs, and sheep.

This document also provides methods and materials for using a composition containing Prevotella (e.g., P. histicola) or Prevotella (e.g., P. histicola) components as a medicament for COPD. In some cases, a composition containing Prevotella (e.g., P. histicola) or Prevotella (e.g., P. histicola) components can be used as a nutritional supplement to supplement a mammal's diet with bacterial organisms having the ability to reduce the severity or development of COPD. Examples of mammals that can be treated as described herein include, without limitation, humans, monkeys, dogs, cats, cows, horses, pigs, and sheep.

This document also provides methods and materials for using a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof as a medicament for COPD. In some cases, a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be used as a nutritional supplement to supplement a mammal's diet with bacterial organisms having the ability to reduce the severity or development of COPD. Examples of mammals that can be treated as described herein include, without limitation, humans, monkeys, dogs, cats, cows, horses, pigs, and sheep.

Any amount of a composition containing Prevotella (e.g., P. histicola) or Prevotella (e.g., P. histicola) components can be administered to a mammal. The dosages of Prevotella (e.g., P. histicola) (e.g., live or killed Prevotella (e.g., P. histicola)) or Prevotella (e.g., P. histicola) components can depend on many factors including the desired results. Typically, the amount of Prevotella (e.g., P. histicola) (e.g., live or killed Prevotella (e.g., P. histicola)) or Prevotella (e.g., P. histicola) components contained within a single dose can be an amount that effectively exhibits anti-inflammatory activity within the mammal. For example, a composition containing live Prevotella (e.g., P. histicola) can be formulated in a dose such that a mammal receives between about 10 and 10¹⁴ live Prevotella (e.g., P. histicola) microorganisms.

Any amount of a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be administered to a mammal. The dosages of live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof components can depend on many factors including the desired results. Typically, the amount of live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof contained within a single dose can be an amount that effectively exhibits anti-inflammatory activity within the mammal. For example, a composition containing live Prevotella (e.g., P. histicola and/or P. melaninogenica) can be formulated in a dose such that a mammal receives between about 10 and 10¹⁴ live Prevotella (e.g., P. histicola and/or P. melaninogenica) microorganisms.

The final pH of a composition containing Prevotella (e.g., P. histicola) (e.g., live or killed Prevotella (e.g., P. histicola)) or Prevotella (e.g., P. histicola) components can be between about 3.5 and about 9.5 (e.g., between about 4.0 and about 9.0; between about 4.5 and about 9.0; between about 4.5 and about 8.5; between about 5.0 and about 8.5; or between about 6.5 and about 8.0). The final pH of a composition containing live Prevotella (e.g., P. histicola and/or P. melaninogenica), dead (or killed) Prevotella (e.g., P. histicola and/or P. melaninogenica), components of Prevotella (e.g., P. histicola and/or P. melaninogenica), vesicles (e.g., OMVs) of Prevotella (e.g., P. histicola and/or P. melaninogenica), or a combination thereof can be between about 3.5 and about 9.5 (e.g., between about 4.0 and about 9.0; between about 4.5 and about 9.0; between about 4.5 and about 8.5; between about 5.0 and about 8.5; or between about 6.5 and about 8.0). To obtain such a pH, the pH of the composition can be adjusted using a pH-adjusting agent, for example. It will be appreciated that pH adjustment can be accomplished with any of a wide variety of acids should the composition have a pH that is too high (e.g., greater than 10.0 before adjustment). Likewise, pH adjustment can be accomplished with any of a wide variety of bases should the composition have a pH that is too low (e.g., less than 3.0 before adjustment).

Non-human animal models of CS-induced emphysema can, in some cases, shed light on mechanisms; however limited information has been available on the role of the lung microbiome. Non-human animal models can require 4-6 months of exposure to CS, which is an arduous task (see, e.g., Wright, J. L., and A. Churg. Expert Rev Respir Med 4: 723-734, 2010), and sometimes the immune responses in animal models do not reproduce the human effector response.

This document also provides a non-human animal model of COPD. Such non-human animal models can be any appropriate species of animal. In some embodiments, a non-human animal model can be a non-human mammal. Examples of non-human animals that can be used to make a model described herein include, without limitation, rodents, rabbits, dogs, cats, cows, pigs, sheep, goats, and non-human primates. For example, in some cases, the non-human animal can be a rodent (e.g., a mouse, a rat, or a guinea pig). In another example, the non-human animal can be a rabbit. In yet another example, the non-human animal can be a non-human primate.

A non-human animal model of COPD can be genetically engineered to alter one or more aspects of the non-human animal's genome, for example, to reproduce a human effector response (see, e.g., Taneja, V., and C. S. David. Immunological reviews 233: 62-78. (2010)). In some cases, a non-human animal model can be designed to express one or more human leukocyte antigens (HLAs) (e.g., serotype DQ8). In some cases, a non-human animal model described herein can be designed to lack the ability to express functional endogenous major histocompatibility complex (MHC) Class II molecules. In some cases, a non-human animal described herein can be designed to lack the ability to express endogenous functional interleukin 17 (IL-17) polypeptides. For example, a non-human animal model of COPD described herein can be designed (a) to express one or more human HLAs (e.g., DQ8), (b) to lack the ability to express functional endogenous class II MHC molecules, and (c) to lack the ability to express functional endogenous IL-17 polypeptides. As used herein, a non-human animal model of COPD provided herein expressing DQ8 and lacking endogenous MHC class II molecules can be notated as “AEo.DQ8” or “DQ8”; a non-human animal model of COPD provided herein lacking functional IL-17 can be notated as “IL-17^{-/-}”; and a non-human animal model of COPD expressing DQ8, lacking expression of functional endogenous MHC class II molecules, and lacking expression of functional endogenous IL-17 can be notated as “DQ8.IL-17^{-/-}”.

In some embodiments, a non-human animal model of COPD provided herein can be an animal that was exposed to cigarette smoke (CS) for a period of time, such as a period of time less than about 6 weeks (e.g., less than about 5 weeks, less than about 4 weeks). In some embodiments, a non-human animal model of COPD provided herein can be an animal that was exposed to cigarette smoke (CS) for about 3 to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks). In some embodiments, a non-human animal model of COPD provided herein can exhibit a loss of lung function (e.g., as compared to a similar animal (e.g., of the same genotype) that was exposed to air instead of cigarette smoke). A loss of lung function can be determined based on any appropriate measure, such as lung compliance (CST; also called pulmonary compliance), lung volume, airway resistance, and elastance, as determined by any appropriate method. In some embodiments, a non-human animal model of COPD provided herein (e.g., a DQ8.IL-17^{-/-0} mouse) that was exposed to CS can have an altered level of expression of one or more genes compared to a similar non-human animal exposed to air instead of CS (see, e.g., Barnes, J. Allergy Clin. Immunol., 138:16-27 (2016)). In some embodiments, a non-human animal model of COPD provided herein (e.g., a DQ8.IL-17^{-/-} mouse) that was exposed to CS can have an increase in expression (e.g., measured by mRNA level) in C-X-C motif chemokine receptor 3 (CXCR3), C-X-C motif chemokine ligand 10 (CXCL10), or both compared to a similar non-human animal exposed to air instead of CS. In some embodiments, a non-human animal model of emphysema provided herein (e.g., a DQ8.IL-17^{-/-} mouse) that was exposed to CS can have a decrease in expression (e.g., measured by mRNA level using any appropriate method) in fibrosis-associated genes, such as collagen a1 (Coll a1), fibronectin (Fib), and transforming growth factor beta (TGFβ) compared to a similar non-human animal exposed to air instead of CS.

Non-human animals (e.g., mice) expressing human HLA-DQ8 and lacking expression of endogenous class II molecules (e.g., AEo.DQ8 mice) when exposed to CS from about 3 weeks to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks) can develop mild to moderate loss of lung function, a pathological feature of COPD, as compared to comparable animals exposed to air (e.g., air-exposed AEo.DQ8 mice), while non-human animals (e.g., mice) expressing human HLA-DQ8, lacking expression of endogenous class II molecules, and lacking expression of endogenous IL-17 polypeptides (e.g., DQ8.IL-17^{-/-} mice) when exposed to CS from about 3 weeks to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks) can develop more pronounced loss of lung function as compared to comparable animals exposed to air (e.g., air-exposed DQ8.IL-17^{-/-} mice). In some cases, non-human animals (e.g., mice) expressing human HLA-DQ8, lacking expression of endogenous class II molecules, and lacking expression of endogenous IL-17 polypeptides (e.g., DQ8.IL-17−/− mice) when exposed to CS from about 3 weeks to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks) can develop more pronounced loss of lung function as compared to similar mice with expression of endogenous IL-17 (e.g., CS-exposed AEo.DQ8 mice).

This document also provides methods for generating non-human animal models of COPD. To form the non-human animal model described herein, a non-human animal can be genetically modified to lack endogenous MHC class II molecules. In some cases, the non-human animal can further be engineered to express one or more human leukocyte antigens (HLAs). In some cases, the non-human animal can be engineered to express HLA serotype DQ8 as described elsewhere (see, e.g., Vassallo, R., et al. Clin Immunol 152: 25-35, 2014, see also U.S. Pat. No. 5,965,787, for example, column 10, lines 25-44, which is incorporated herein by reference in its entirety). Any appropriate genetic modification techniques can be used. For example, transgenic techniques, such that all the somatic cells of the non-human animal express human DQ8, can be employed (see, e.g., Vassallo, R., et al. Clin Immunol 152: 25-35, 2014). Further, additional genes may be knocked out using any appropriate technique. For example, nucleic acid encoding an IL-17 polypeptide can be rendered non-functional (e.g., “knocked out” or mutated) using any appropriate method. For example, a knock-out construct designed to disrupt an endogenous nucleic acid sequence can be used. A disruption can be positioned at any appropriate site in an endogenous nucleic acid sequence. Examples of disruptions include, without limitation, deletions in the endogenous sequence and insertions of heterologous nucleic acid sequences into the endogenous sequence. Examples of insertions can include, without limitation, artificial splice acceptors coupled to stop codons or splice donors coupled to fusion partners such as GFP. A knock-out construct can contain sequences that are homologous to the endogenous nucleic acid sequence or to sequences that are adjacent to the endogenous nucleic acid sequence. In some cases, a knock-out construct can contain a nucleic acid sequence encoding a selection marker (e.g., antibiotic resistance, a fluorescent reporter (e.g., GFP or YFP), or an enzyme (e.g., (3-galactosidase)) operatively linked to a regulatory sequence (e.g., a promoter). A knock-out construct can include other nucleic acid sequences such as recombination sequences (e.g., loxP sequences), splice acceptor sequences, splice donor sequences, transcription start sequences, and transcription stop sequences. Disruptions in the endogenous nucleic acid sequence can result in reduced expression of the gene or non-functional truncations or fusions of the encoded polypeptide.

In some embodiments, a method of generating a non-human animal model of COPD can include providing an AEo.DQ8 non-human animal (e.g., a AEo.DQ8 mouse) and subjecting the non-human animal to cigarette smoke for about 3 weeks to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks). In some embodiments, a method of generating a non-human animal model of COPD can include providing a DQ8.IL-17^{-/-} non-human animal (e.g., a DQ8.IL-17^{-/-} mouse) and subjecting the non-human animal to cigarette smoke for about 3 weeks to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks). Subjecting an animal to cigarette smoke for about 3 weeks to about 6 weeks (e.g., about 3 to about 5 weeks, about 3 to about 4 weeks, about 4 to about 5 weeks, about 4 to about 6 weeks, or about 5 to about 6 weeks, about 4 weeks, or about 5 weeks) can be achieved by any appropriate method. In some embodiments, subjecting an animal to cigarette smoke for about 3 weeks to about 6 weeks can include exposing mice to smoke from about two cigarettes about every 10 minutes for about 3 hours per day about 5 days per week. In some embodiments, subjecting an animal to cigarette smoke for about 3 weeks to about 6 weeks can include sufficient exposure to cigarette smoke to result in blood nicotine levels of about 45 to about 185 ng/mL (e.g., about 50 to about 185 ng/mL, about 100 to about 185 ng/mL, about 150 to about 185 ng/mL, about 45 to about 75 ng/mL, about 45 to about 100 ng/mL, about 45 to about 150 ng/mL, about 50 to about 150 ng/mL, or about 75 to about 125 ng/mL) at the end of the exposure period.

In some embodiments, a method of generating a non-human animal model of COPD can include providing an AEo.DQ8 non-human animal (e.g., a AEo.DQ8 mouse) and subjecting the non-human animal to cigarette smoke for less than about 6 weeks (e.g., less than about 5 weeks, less than about 4 weeks). In some embodiments, a method of generating a non-human animal model of COPD can include providing a DQ8.IL-17^{-/-} non-human animal (e.g., a DQ8.IL-17^{-/-} mouse) and subjecting the non-human animal to cigarette smoke for less than about 6 weeks (e.g., less than about 5 weeks, less than about 4 weeks). Subjecting an animal to cigarette smoke for less than about 6 weeks (e.g., less than about 5 weeks, less than about 4 weeks) can be achieved by any appropriate method. In some embodiments, subjecting an animal to cigarette smoke for less than about 6 weeks can include exposing mice to smoke from about two cigarettes about every 10 minutes for about 3 hours per day about 5 days per week. In some embodiments, subjecting an animal to cigarette smoke for less than about 6 weeks can include sufficient exposure to cigarette smoke to result in blood nicotine levels of about 45 to about 185 ng/mL (e.g., about 50 to about 185 ng/mL, about 100 to about 185 ng/mL, about 150 to about 185 ng/mL, about 45 to about 75 ng/mL, about 45 to about 100 ng/mL, about 45 to about 150 ng/mL, about 50 to about 150 ng/mL, or about 75 to about 125 ng/mL) at the end of the exposure period.

The invention will be further described in the following examples, which do not limit the scope of the invention described in the claims.

### EXEMPLARY EMBODIMENTS

Embodiment 1 is a method for treating chronic obstructive pulmonary lung disease (COPD) in a mammal, wherein said method comprises administering a composition comprising live or killed Prevotella to the mammal.

Embodiment 2 is the method of embodiment 1, wherein the composition comprises live Prevotella.

Embodiment 3 is the method of embodiment 1, wherein the composition comprises killed Prevotella and no live Prevotella.

Embodiment 4 is the method of any one of embodiments 1-3, wherein the mammal is a human.

Embodiment 5 is the method of any one of embodiments 1-3, wherein the mammal is a rodent.

Embodiment 6 is the method of any one of embodiments 1-5, wherein administering comprises oral administration.

Embodiment 7 is the method of any one of embodiments 1-6, wherein the composition is a pill, tablet, or capsule.

Embodiment 8 is the method of any one of embodiments 1-7, wherein the composition is a pill, tablet, or capsule configured to deliver the Prevotella to the intestines of the mammal.

Embodiment 9 is the method of any one of embodiments 1-5, wherein administering comprises administration to the respiratory system.

Embodiment 10 is the method of any one of embodiments 1-5 or 9, wherein the composition is administered using a nasal spray, an inhaler, or a nebulizer.

Embodiment 11 is the method of any one of embodiments 1-10, wherein the severity of a symptom of said COPD is reduced following the administering.

Embodiment 12 is the method of embodiment 11, wherein the severity of the symptom of said COPD is reduced by greater than about 25 percent following the administering.

Embodiment 13 is the method of embodiment 11, wherein the severity of the symptom of said COPD is reduced by greater than about 50 percent following the administering.

Embodiment 14 is the method of embodiment 11, wherein the severity of the symptom of said COPD is reduced by greater than about 75 percent following the administering.

Embodiment 15 is the method of any one of embodiments 11-14, wherein lung compliance of said mammal is decreased following said administering step.

Embodiment 16 is the method of embodiment 15, wherein lung compliance after the administering is decreased by at least about 5% as compared to before the administering.

Embodiment 17 is the method of embodiment 15, wherein lung compliance after the administering is decreased by at least about 10% as compared to before the administering.

Embodiment 18 is the method of embodiment 15, wherein lung compliance after the administering is decreased by at least about 15% as compared to before the administering.

Embodiment 19 is the method of any one of embodiments 1-18, further comprising measuring one or more parameters of lung function before the administering, and measuring the same one or more parameters of lung function following the administering.

Embodiment 20 is the method of embodiment 19 wherein at least one of the one or more parameters of lung function measured following the administering is improved compared to before the administering.

Embodiment 21 is the method of any one of embodiments 19-20, wherein the one or more parameters of lung function comprise lung compliance, lung volume, airway resistance, elastance, or a combination thereof.

Embodiment 22 is the method of any one of embodiments 1-21, wherein the method comprises identifying said mammal as having COPD prior to said administering step.

Embodiment 23 is the method of any one of embodiments 1-22, wherein the method comprises identifying said mammal as having emphysema prior to said administering step.

Embodiment 24 is the method of any one of embodiments 1-23, wherein representative cells of the Prevotella are deposited as NRRL accession number B-50329.

Embodiment 25 is the method of any one of embodiments 1-24, wherein the composition comprises about 1×10³ to about 1×10¹⁴ CFU of Prevotella.

Embodiment 26 is the method of any one of embodiments 1-24, wherein the composition comprises about 1×10⁵ to about 1×10¹² CFU of Prevotella.

Embodiment 27 is the method of any one of embodiments 1-24, wherein the composition comprises about 1×10⁸ to about 1×10¹⁰ CFU of Prevotella.

Embodiment 28 is the method of any one of embodiments 1-27, wherein the composition is administered once per day.

Embodiment 29 is the method of any one of embodiments 1-27, wherein the composition is administered twice per day.

Embodiment 30 is the method of any one of embodiments 1-27, wherein the composition is administered three times per day.

Embodiment 31 is the method of any one of embodiments 1-30, wherein the composition is administered for a period of at least about 4 weeks.

Embodiment 32 is the method of any one of embodiments 1-30, wherein the composition is administered for a period of at least about 8 weeks.

Embodiment 33 is the method of any one of embodiments 1-32, wherein the Prevotella comprises Prevotella histicola.

Embodiment 34 is a non-human mammalian model of chronic obstructive pulmonary disease (COPD), wherein the somatic cells of said non-human mammalian model comprise nucleic acid encoding one or more human HLA serotypes, and wherein the somatic cells of said non-human mammalian model lack expression of endogenous MHC class II molecules and endogenous IL-17 polypeptides, and wherein, when exposed to cigarette smoke for about 2 to about 6 weeks, said model has a decrease in lung function as compared to a compared to a comparable model not exposed to cigarette smoke.

Embodiment 35 is the non-human mammalian model of embodiment 34, wherein the human HLA serotype is DQ8.

Embodiment 36 is 36 is the non-human mammalian model of embodiment 34 or embodiment 35, wherein the model is an IL-17^{-/-} knock-out mammal.

Embodiment 37 is the non-human mammalian model of any one of embodiments 34-36, wherein the mammal is a rodent.

Embodiment 38 is the non-human mammalian model of any one of embodiments 34-37, wherein the mammal is a mouse.

Embodiment 39 is the non-human mammalian model of any one of embodiments 34-38, wherein the mammal has a blood nicotine level of about 45 to about 185 ng/mL.

Embodiment 40 is the non-human mammalian model of any one of embodiments 34-39, wherein the mammal has a blood nicotine level of about 50 to about 150 ng/mL.

Embodiment 41 is the non-human mammalian model of any one of embodiments 34-40, wherein the mammal has an increased level of expression in CXCR3, CXCL10, or both, as compared to a comparable mammal that is not a non-human animal model of COPD.

Embodiment 42 is the non-human mammalian model of any one of embodiments 34-41, the mammal has a decreased level of expression in one or more fibrosis-associated genes, as compared to a comparable mammal that is not a non-human animal model of COPD.

Embodiment 43 is the non-human mammalian model of embodiment 42, wherein at least one of the fibrosis-associated genes is selected from the group consisting of collagen a1, fibronectin, and transforming growth factor beta.

Embodiment 44 is the non-human mammalian model of any one of embodiments 34-43, wherein said decrease in lung function is measured using one or more parameters of lung function.

Embodiment 45 is the non-human mammalian model of embodiment 44, wherein the one or more parameters of lung function comprise lung compliance, lung volume, airway resistance, elastance, or a combination thereof.

Embodiment 46 is a method of producing a non-human mammalian model for chronic obstructive pulmonary disease (COPD), the method comprising:
providing non-human mammal, wherein the somatic cells of said non-human mammalian comprise nucleic acid encoding one or more human HLA serotypes, and wherein the somatic cell of said non-human mammalian model lack expression of endogenous MHC class II molecules and endogenous IL-17 polypeptide; and
exposing the mammal to cigarette smoke for about 3 to about 6 weeks,
thereby producing a non-human animal model for COPD.

Embodiment 47 is the method of embodiment 46, wherein the human HLA serotype is DQ8.

Embodiment 48 is the method of embodiment 46 or embodiment 47, wherein the mammal is IL-17^{-/-}.

Embodiment 49 is the method of any one of embodiments 46-48, wherein the mammal is a rodent.

Embodiment 50 is the method of any one of embodiments 46-49, wherein the mammal is a mouse.

Embodiment 51 is the method of any one of embodiments 46-50, wherein said exposing comprises exposing the mammal to about 2 cigarettes about 10 minutes for about 3 hours per day about 5 days per week.

Embodiment 52 is the method of any one of embodiments 46-51, wherein said exposing comprises sufficient exposure to cigarette smoke to result in a blood nicotine level of about 45 to about 185 ng/mL.

Embodiment 53 is the method of any one of embodiments 46-51, wherein said exposing comprises sufficient exposure to cigarette smoke to result in a blood nicotine level of about 50 to about 150 ng/mL.

Embodiment 54 is the method of any one of embodiments 46-53, wherein following the exposing, the mammal has an increased level of expression in CXCR3, CXCL10, or both, as compared to a similar mammal exposed to air instead of cigarette smoke.

Embodiment 55 is the method of any one of embodiments 46-54, wherein following the exposing, the mammal has a decreased level of expression in one or more fibrosis-associated genes, as compared to a similar mammal exposed to air instead of cigarette smoke.

Embodiment 56 is the method of embodiment 55, wherein at least one of the fibrosis-associated genes is selected from the group consisting of collagen a1, fibronectin, and transforming growth factor beta.

Embodiment 57 is the method of any one of embodiments 46-55, wherein following the exposing, the mammal has a decrease in lung function as compared to a similar mammal exposed to air instead of cigarette smoke.

Embodiment 58 is the method of embodiment 57, wherein said decrease in lung function is measured using one or more parameters of lung function.

Embodiment 59 is the method of embodiment 58, wherein the one or more parameters of lung function comprise lung compliance, lung volume, airway resistance, elastance, or a combination thereof.

Embodiment 60 is a non-human mammalian model of COPD produced by the method of any one of embodiments 46-59.

Embodiment 61 is a method for treating a lung condition in a mammal, wherein said method comprises administering a composition comprising live or killed Prevotella to the mammal.

Embodiment 62 is the method of embodiment 61, wherein the composition comprises live Prevotella.

Embodiment 63 is the method of embodiment 61, wherein the composition comprises killed Prevotella and no live Prevotella.

Embodiment 64 is the method of any one of embodiments 61-63, wherein the mammal is a human.

Embodiment 65 is the method of any one of embodiments 61-63, wherein the mammal is a rodent.

Embodiment 66 is the method of any one of embodiments 61-65, wherein administering comprises oral administration.

Embodiment 67 is the method of any one of embodiments 61-66, wherein the composition is a pill, tablet, or capsule.

Embodiment 68 is the method of any one of embodiments 61-67, wherein the composition is a pill, tablet, or capsule configured to deliver the Prevotella to the intestines of the mammal.

Embodiment 69 is the method of any one of embodiments 61-65, wherein administering comprises administration to the respiratory system.

Embodiment 70 is the method of any one of embodiments 61-65 or 69, wherein the composition is administered using a nasal spray, an inhaler, or a nebulizer.

Embodiment 71 is the method of any one of embodiments 61-70, wherein the severity of a symptom of said lung condition is reduced following the administering.

Embodiment 72 is the method of embodiment 71, wherein the severity of the symptom of said lung condition is reduced by greater than about 25 percent following the administering.

Embodiment 73 is the method of embodiment 71, wherein the severity of the symptom of said lung condition is reduced by greater than about 50 percent following the administering.

Embodiment 74 is the method of embodiment 71, wherein the severity of the symptom of said lung condition is reduced by greater than about 75 percent following the administering.

Embodiment 75 is the method of any one of embodiments 71-74, wherein lung compliance of said mammal is decreased following said administering step.

Embodiment 76 is the method of embodiment 75, wherein lung compliance after the administering is decreased by at least about 5% as compared to before the administering.

Embodiment 77 is the method of embodiment 75, wherein lung compliance after the administering is decreased by at least about 10% as compared to before the administering.

Embodiment 78 is the method of embodiment 75, wherein lung compliance after the administering is decreased by at least about 15% as compared to before the administering.

Embodiment 79 is the method of any one of embodiments 61-78, further comprising measuring one or more parameters of lung function before the administering, and measuring the same one or more parameters of lung function following the administering.

Embodiment 80 is the method of embodiment 79 wherein at least one of the one or more parameters of lung function measured following the administering is improved compared to before the administering.

Embodiment 81 is the method of any one of embodiments 79-80, wherein the one or more parameters of lung function comprise lung compliance, lung volume, airway resistance, elastance, or a combination thereof.

Embodiment 82 is the method of any one of embodiments 61-81, wherein the method comprises identifying said mammal as having the lung condition prior to said administering step.

Embodiment 83 is the method of any one of embodiments 61-82, wherein the method comprises identifying said mammal as having pneumonia prior to said administering step.

Embodiment 84 is the method of any one of embodiments 61-83, wherein representative cells of the Prevotella are deposited as NRRL accession number B-50329.

Embodiment 85 is the method of any one of embodiments 61-84, wherein the composition comprises about 1×10³ to about 1×10¹⁴ CFU of Prevotella.

Embodiment 86 is the method of any one of embodiments 61-84, wherein the composition comprises about 1×10⁵ to about 1×10¹² CFU of Prevotella.

Embodiment 87 is the method of any one of embodiments 61-84, wherein the composition comprises about 1×10⁸ to about 1×10¹⁰ CFU of Prevotella.

Embodiment 88 is the method of any one of embodiments 61-87, wherein the composition is administered once per day.

Embodiment 89 is the method of any one of embodiments 61-87, wherein the composition is administered twice per day.

Embodiment 90 is the method of any one of embodiments 61-87, wherein the composition is administered three times per day.

Embodiment 91 is the method of any one of embodiments 61-90, wherein the composition is administered for a period of at least about 4 weeks.

Embodiment 92 is the method of any one of embodiments 61-90, wherein the composition is administered for a period of at least about 8 weeks.

Embodiment 93 is the method of any one of embodiments 61-92, wherein the Prevotella comprises Prevotella histicola.

Embodiment 94 is the method of any one of embodiments 61-93, wherein the lung condition is selected from the group consisting of chronic obstructive pulmonary disease (COPD), asthma, pulmonary fibrosis, sarcoidosis, disease caused by infection, and combinations thereof.

Embodiment 95 is the method of embodiment 94, wherein the disease caused by infection is selected from the group consisting of a viral infection, a bacterial infection, a fungal infection, a parasitic infection, and combinations thereof.

Embodiment 96 is the method of embodiment 95, wherein the viral infection is selected from the group consisting of viral pneumonia, viral bronchitis, influenza, coronavirus infection, adenovirus infection, syncytial virus infection, rhinovirus infection, and combinations thereof.

Embodiment 97 is the method of embodiment 95, wherein the bacterial infection is selected from the group consisting of Streptococcus infection, Staphylococcus infection, Chlamydophila infection, Mycoplasma infection, Haemophilus infection, Corynebacterium infection, Mycobacterium infection, Legionella infection, and combinations thereof.

Embodiment 98 is the method of any one of embodiments 61-93, wherein the lung condition comprises pneumonia.

Embodiment 99 is a method for treating a lung condition in a mammal, wherein said method comprises administering a composition comprising live or killed Prevotella melaninogenica to the mammal.

Embodiment 100 is the method of embodiment 99, wherein the composition comprises live Prevotella melaninogenica.

Embodiment 101 is the method of embodiment 99, wherein the composition comprises killed Prevotella melaninogenica and no live Prevotella melaninogenica.

Embodiment 102 is the method of any one of embodiments 99-101, wherein the mammal is a human.

Embodiment 103 is the method of any one of embodiments 99-101, wherein the mammal is a rodent.

Embodiment 104 is the method of any one of embodiments 99-103, wherein administering comprises oral administration.

Embodiment 105 is the method of any one of embodiments 99-104, wherein the composition is a pill, tablet, or capsule.

Embodiment 106 is the method of any one of embodiments 99-105, wherein the composition is a pill, tablet, or capsule configured to deliver the Prevotella melaninogenica to the intestines of the mammal.

Embodiment 107 is the method of any one of embodiments 99-103, wherein administering comprises administration to the respiratory system.

Embodiment 108 is the method of any one of embodiments 99-103 or 107, wherein the composition is administered using a nasal spray, an inhaler, or a nebulizer.

Embodiment 109 is the method of any one of embodiments 99-108, wherein the severity of a symptom of said lung condition is reduced following the administering.

Embodiment 110 is the method of embodiment 109, wherein the severity of the symptom of said lung condition is reduced by greater than about 25 percent following the administering.

Embodiment 111 is the method of embodiment 109, wherein the severity of the symptom of said lung condition is reduced by greater than about 50 percent following the administering.

Embodiment 112 is the method of embodiment 109, wherein the severity of the symptom of said lung condition is reduced by greater than about 75 percent following the administering.

Embodiment 113 is the method of any one of embodiments 109-112, wherein lung compliance of said mammal is decreased following said administering step.

Embodiment 114 is the method of embodiment 113, wherein lung compliance after the administering is decreased by at least about 5% as compared to before the administering.

Embodiment 115 is the method of embodiment 113, wherein lung compliance after the administering is decreased by at least about 10% as compared to before the administering.

Embodiment 116 is the method of embodiment 113, wherein lung compliance after the administering is decreased by at least about 15% as compared to before the administering.

Embodiment 117 is the method of any one of embodiments 99-116, further comprising measuring one or more parameters of lung function before the administering, and measuring the same one or more parameters of lung function following the administering.

Embodiment 118 is the method of embodiment 117, wherein at least one of the one or more parameters of lung function measured following the administering is improved compared to before the administering.

Embodiment 119 is the method of any one of embodiments 117-118, wherein the one or more parameters of lung function comprise lung compliance, lung volume, airway resistance, elastance, or a combination thereof.

Embodiment 120 is the method of any one of embodiments 99-119, wherein the method comprises identifying said mammal as having the lung condition prior to said administering step.

Embodiment 121 is the method of any one of embodiments 99-120, wherein the method comprises identifying said mammal as having pneumonia prior to said administering step.

Embodiment 122 is the method of any one of embodiments 99-121, wherein the method comprises identifying said mammal as having COPD prior to said administering step.

Embodiment 123 is the method of any one of embodiments 99-122, wherein the composition comprises about 1×10³ to about 1×10¹⁴ CFU of Prevotella melaninogenica.

Embodiment 124 is the method of any one of embodiments 99-122, wherein the composition comprises about 1×10⁵ to about 1×10¹² CFU of Prevotella melaninogenica.

Embodiment 125 is the method of any one of embodiments 99-122, wherein the composition comprises about 1×10⁸ to about 1×10¹⁰ CFU of Prevotella melaninogenica.

Embodiment 126 is the method of any one of embodiments 99-125, wherein the composition is administered once per day.

Embodiment 127 is the method of any one of embodiments 99-125, wherein the composition is administered twice per day.

Embodiment 128 is the method of any one of embodiments 99-125, wherein the composition is administered three times per day.

Embodiment 129 is the method of any one of embodiments 99-128, wherein the composition is administered for a period of at least about 4 weeks.

Embodiment 130 is the method of any one of embodiments 99-128, wherein the composition is administered for a period of at least about 6 weeks.

Embodiment 131 is the method of any one of embodiments 99-128, wherein the composition is administered for a period of at least about 8 weeks.

Embodiment 132 is the method of any one of embodiments 99-131, wherein the lung condition is selected from the group consisting of chronic obstructive pulmonary disease (COPD), asthma, pulmonary fibrosis, sarcoidosis, disease caused by infection, and combinations thereof.

Embodiment 133 is the method of embodiment 132, wherein the disease caused by infection is selected from the group consisting of a viral infection, a bacterial infection, a fungal infection, a parasitic infection, and combinations thereof.

Embodiment 134 is the method of embodiment 133, wherein the viral infection is selected from the group consisting of viral pneumonia, viral bronchitis, influenza, coronavirus infection, adenovirus infection, syncytial virus infection, rhinovirus infection, and combinations thereof.

Embodiment 135 is the method of embodiment 133, wherein the bacterial infection is selected from the group consisting of Streptococcus infection, Staphylococcus infection, Chlamydophila infection, Mycoplasma infection, Haemophilus infection, Corynebacterium infection, Mycobacterium infection, Legionella infection, and combinations thereof.

Embodiment 136 is the method of any one of embodiments 99-131, wherein the lung condition comprises pneumonia.

Embodiment 137 is a method for treating a lung condition in a mammal, wherein said method comprises administering a composition comprising vesicles of a Prevotella species.

Embodiment 138 is the method of embodiment 137, wherein the mammal is a human.

Embodiment 139 is the method of embodiment 137, wherein the mammal is a rodent.

Embodiment 140 is the method of embodiment 137, wherein administering comprises oral administration.

Embodiment 141 is the method of any one of embodiments 137-140, wherein the composition is a pill, tablet, or capsule.

Embodiment 142 is the method of any one of embodiments 137-141, wherein the composition is a pill, tablet, or capsule configured to deliver the vesicles of the Prevotella species to the intestines of the mammal.

Embodiment 143 is the method of any one of embodiments 137-139, wherein administering comprises administration to the respiratory system.

Embodiment 144 is the method of any one of embodiments 137-139 or 143, wherein the composition is administered using a nasal spray, an inhaler, or a nebulizer.

Embodiment 145 is the method of any one of embodiments 137-144, wherein the severity of a symptom of said lung condition is reduced following the administering.

Embodiment 146 is the method of embodiment 145, wherein the severity of the symptom of said lung condition is reduced by greater than about 25 percent following the administering.

Embodiment 147 is the method of embodiment 145, wherein the severity of the symptom of said lung condition is reduced by greater than about 50 percent following the administering.

Embodiment 148 is the method of embodiment 145, wherein the severity of the symptom of said lung condition is reduced by greater than about 75 percent following the administering.

Embodiment 149 is the method of any one of embodiments 145-148, wherein lung compliance of said mammal is decreased following said administering step.

Embodiment 150 is the method of embodiment 149, wherein lung compliance after the administering is decreased by at least about 5% as compared to before the administering.

Embodiment 151 is the method of embodiment 149, wherein lung compliance after the administering is decreased by at least about 10% as compared to before the administering.

Embodiment 152 is the method of embodiment 149, wherein lung compliance after the administering is decreased by at least about 15% as compared to before the administering.

Embodiment 153 is the method of any one of embodiments 137-152, further comprising measuring one or more parameters of lung function before the administering, and measuring the same one or more parameters of lung function following the administering.

Embodiment 154 is the method of embodiment 153 wherein at least one of the one or more parameters of lung function measured following the administering is improved compared to before the administering.

Embodiment 155 is the method of any one of embodiments 153-154, wherein the one or more parameters of lung function comprise lung compliance, lung volume, airway resistance, elastance, or a combination thereof.

Embodiment 156 is the method of any one of embodiments 137-155, wherein the method comprises identifying said mammal as having the lung condition prior to said administering step.

Embodiment 157 is the method of any one of embodiments 137-156, wherein the method comprises identifying said mammal as having pneumonia prior to said administering step.

Embodiment 158 is the method of any one of embodiments 137-157, wherein representative cells of the Prevotella species are deposited as NRRL accession number B-50329.

Embodiment 159 is the method of any one of embodiments 137-158, wherein the composition is administered once per day.

Embodiment 160 is the method of any one of embodiments 137-158, wherein the composition is administered twice per day.

Embodiment 161 is the method of any one of embodiments 137-158, wherein the composition is administered three times per day.

Embodiment 162 is the method of any one of embodiments 137-161, wherein the composition is administered for a period of at least about 4 weeks.

Embodiment 163 is the method of any one of embodiments 137-161, wherein the composition is administered for a period of at least about 8 weeks.

Embodiment 164 is the method of any one of embodiments 137-163, wherein the Prevotella species comprises Prevotella histicola.

Embodiment 165 is the method of any one of embodiments 137-164, wherein the Prevotella species comprises Prevotella melaninogenica.

Embodiment 166 is the method of any one of embodiments 137-165, wherein the Prevotella species comprises Prevotella histicola and Prevotella melaninogenica.

Embodiment 167 is the method of any one of embodiments 137-165, wherein the lung condition is selected from the group consisting of chronic obstructive pulmonary disease (COPD), asthma, pulmonary fibrosis, sarcoidosis, disease caused by infection, and combinations thereof.

Embodiment 168 is the method of embodiment 166, wherein the disease caused by infection is selected from the group consisting of a viral infection, a bacterial infection, a fungal infection, a parasitic infection, and combinations thereof.

Embodiment 169 is the method of embodiment 167, wherein the viral infection is selected from the group consisting of viral pneumonia, viral bronchitis, influenza, coronavirus infection, adenovirus infection, syncytial virus infection, rhinovirus infection, and combinations thereof.

Embodiment 170 is the method of embodiment 167, wherein the bacterial infection is selected from the group consisting of Streptococcus infection, Staphylococcus infection, Chlamydophila infection, Mycoplasma infection, Haemophilus infection, Corynebacterium infection, Mycobacterium infection, Legionella infection, and combinations thereof.

Embodiment 171 is the method of any one of embodiments 137-165, wherein the lung condition comprises pneumonia.

### EXAMPLES

### Example 1

A human diagnosed with COPD or displaying one or more symptoms thereof (e.g., emphysema) is treated with a composition containing live P. histicola. The composition containing live P. histicola is administered orally to the human in a dose of about 1×10³ to about 1×10¹⁴ CFU once a day for 8 weeks. The human is monitored to confirm reduction of one or more symptoms of COPD.

### Example 2

A human diagnosed with COPD or displaying one or more symptoms thereof (e.g., emphysema) is treated with a composition containing live P. histicola. The composition containing live P. histicola is administered nasally to the human in a dose of about 1×10³ to about 1×10¹⁴ CFU once a day for 8 weeks. The human is monitored to confirm reduction of one or more symptoms of COPD.

### Example 3

A model of cigarette smoke (CS)-induced emphysema was generated using DQ8 mice. Exposure to CS for 4-5 weeks changed the lung immunity by enhancing pro-inflammatory cytokines such as IL-17 and induced mild emphysema. The effect of IL-17 in CS-induced emphysema was investigated using DQ8.IL-17^{-/-} mice. Exposure to CS for 4 weeks led to severe emphysema in these mice, indicating a role of IL-17 in protection. COPD lungs can have altered microbiota, in some cases with selective deletion of Prevotella species. These results indicate that airway microbiota may be involved in directing the differentiation of inflammatory cytokines, which in turn promote influx of inflammatory cells and COPD.

### Example 4

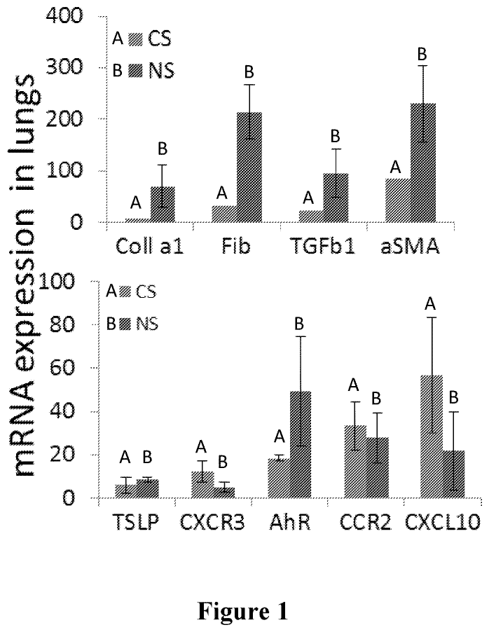

To investigate the role of cytokines in lung pathology, DQ8 mice were exposed to CS or air for 5 weeks, and the immune response in the lungs was determined by reverse transcriptase polymerase chain reaction (rtPCR) (FIG. 1). FIG. 1 is a bar plot of mRNA expression in the lungs of mice exposed to CS (red bars) or air (blue bars). Mice exposed to CS exhibited an increase in CXCR3 and CXCL10, similar to that known in smokers (see, e.g., Saetta, et al., Am. J. Respir. Crit. Care Med., 165:1404-1409 (2002)) with a reduced expression of fibrosis-associated genes, Coll a1, fibronectin, and TGFβ.

### Example 5

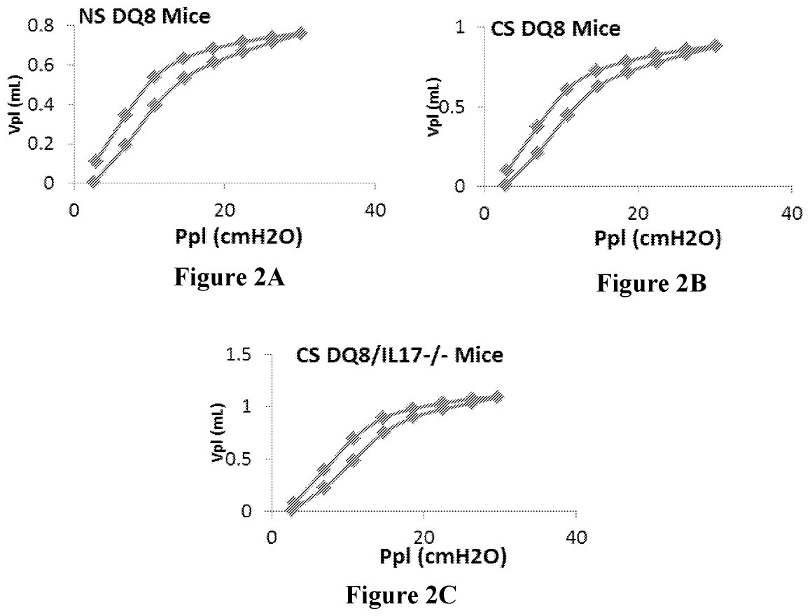

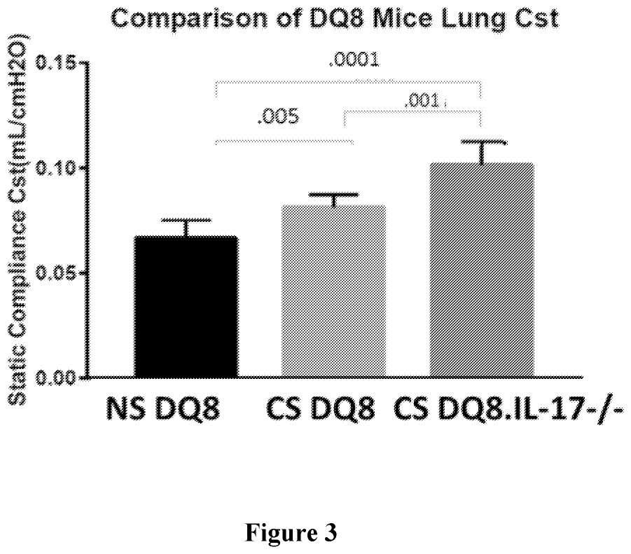

The role of IL-17 in emphysema was investigated by generating AEo.DQ8 mice deficient in IL-17, (DQ8.IL-17^{-/-}). DQ8 and DQ8.IL-17^{-/-} mice were exposed to CS for 4 weeks. DQ8 mice exposed to air (NS) were used as controls (FIGS. 2A, 2B, and 2C). Lung function in mice was measured by determining lung compliance (CST), by performing a lung mechanics scan in live animals. A pressure-volume (PV) curve was studied in three groups of mice, and the data was compiled as depicted (NS DQ8 mice (FIG. 2A), CS DQ8 mice (FIG. 2B), N=7 each and DQ8.IL-17^{-/-}, CS mice (FIG. 2C), N=4). These results demonstrate that humanized mice exposed to CS developed emphysema, a key feature of COPD. These results also demonstrate a protective role of IL-17 in COPD, as in the absence of IL-17, there was an increase in lung compliance. As shown in FIG. 3, a statistically significant increase in lung compliance was observed in the CS-exposed DQ8 as well as DQ8.IL-17^{-/-} mice relative to the control “non-smoker” (NS) DQ8 mice.

Without being bound by any particular theory, it is believed that the role of IL-17 in COPD could be 1) a compensatory mechanism or 2) that dysbiosis leads to an increase in IL-17.

The DQ8.IL-17^{-/-} model was different from other models in that COPD developed after a short period of CS exposure (e.g., after about 4 weeks), as compared to the 16-24 weeks reported for other models (see, e.g., Wright, J. L., and A. Churg. Expert. Rev. Respir. Med. 4: 723-734. (2010)). In addition, the DQ8.IL-17^{-/-} model exhibited an effector arm of the immune response restricted by human DQ8 molecules and mimicked humans in immune response to various antigens and autoimmune pathologies.

### Example 6

As shown in Example 5, emphysema was developed in smoker DQ8.IL-17^{-/-} mice, a model in which an exposure to CS for 3-4 weeks led to chronic lung inflammation with alveolar destruction and emphysema. Oral treatment with P. histicola is performed to confirm the modulation of emphysema in CS-exposed DQ8.IL-17^{-/-} mice through the generation of eubiosis and reduction of lung inflammation. Briefly, treatment of CS-induced emphysema in DQ8.IL-17^{-/-} mice with P. histicola or P. melaninogenica is performed as is a determination of changes in lung function. DQ8.IL-17^{-/-} mice exposed to air instead of CD are used as control. Improvement in lung function in treated mice is measured using static lung compliance.

### Example 7

Patients with COPD have low abundance of Prevotella, one of the most abundant genera in oral, lung, and gut mucosa. It is determined whether CS-induced emphysema can be treated by an oral mucosa dominant human commensal.

DQ8.IL-17^{-/-} mice are exposed to CS daily for 2 weeks, and the cohort is randomly assigned to 4 groups:

Group 1—media treatment (Control),

Group 2—sham control,

Group 3—oral treatment with P. histicola, and

Group 4—oral treatment with P. melaninogenica.

As shown in Example 5, FIGS. 2A-2C, and FIG. 3, within 4 weeks of exposure to CS, mice exhibited a significant loss of lung function. By 2 weeks of exposure, structural changes appeared to start.

This duration of exposure is chosen because 4 weeks is the time required to reproducibly and effectively induce physiologic abnormalities of emphysema in the DQ8.IL-17^{-/-} mice as shown in Example 5. Lung function mechanics is a terminal event, hence after CS exposure for 2 weeks, the experimental groups are treated orally for 2 weeks on alternate days with live bacteria (10\hat{ }9 CFU/100 μL) in bacterial culture. Mice are continued to be exposed to CS with total exposure of 4 weeks. Control age-matched mice are sham-smoked: they are placed in cages adjacent to the cigarette smoke chamber. About 7-10 mice are included in each group for lung function.

Chronic CS exposure is performed in a smoking chamber (Teague enterprises, CA) that exposes mice to regulated concentrations of CS inhalation generated by 3R4F Kentucky research cigarettes (2 cigarettes every 10 minutes for 3 h/day on a daily basis for 5 days/week, as described elsewhere (see, e.g., Kroening, et al., Journal of Immunology, 181:1536-1547 (2008)). In this system, mice are exposed to a mixture of mainstream and side-stream cigarette smoke, and this enables sufficient exposure that results in generation of murine blood nicotine levels analogous to levels attained in heavy human smokers (range of about 45-181 ng/mL) (see, e.g., Kroening, et al., Journal of Immunology, 181:1536-1547 (2008); and Jarvik, et al., Pharmacol. Biochem. Behay., 66:553-558 (2000)).

Mice are monitored daily and weighed alternative days to determine their condition.

Lung function analysis is performed at the termination of experiment. The lung function of CS exposed and control mice is performed using the flexiVent® system. This allows determination of lung compliance, volume, and airway resistance. Baseline measurements of respiratory system resistance, compliance, and elastance are performed during a 2-s breath hold with a 2.5-Hz sinusoidal oscillation using the single-compartment model (see, e.g., Ewart, S., et al. J Appl Physiol (1985) 79: 560-566.). The effect of 4 week exposure to cigarette smoke is determined using all 4 groups of mice. At the end of the exposure period, static lung compliance, airway and tissue resistance is performed under spontaneous ventilation.

The measurements are performed to confirm that P. histicola and P. melaninogenica suppress emphysema.

### Example 8

CS leads to dysbiosis of the lung microbiome, which can change the local immune response causing pro-inflammatory cytokines to accumulate. Structural and molecular changes in lungs exposed to CS are analyzed by conducting a number of complimentary strategies.

To determine morphologic airway and distal lung changes, mice are be treated as stated in Example 7. At the termination of the experiment at week 4, mice are sacrificed. Lungs are inflated with 0.5 mL of 10% neutral-buffered formalin, and fixed overnight. The fixed lung tissue is embedded in paraffin, and sectioned and stained with hematoxylin and eosin staining (H&E staining) to determine the presence of morphological features of emphysema (e.g., expansion of inter-alveolar walls and determination of mean linear intercept (see, e.g, Hasleton, P. S. Pathol Eur 11: 211-218, (1976))). Four to five vessels are assessed per mouse. Briefly, for each vessel, two characteristics of the perivascular infiltrate are assessed. The perimeter score represents percentage of vessel perimeter surrounded by cells. A score of 1 represents 5-20% of vessel perimeter surrounded by cells; a score of 2 represents 25-45% of vessel surrounded by cells; a score of 3 indicates for 50-75%; and a score of 4 represents 75-100% of vessel surrounded by cells.

In another set of mice, one lung is frozen at −80° C. and is later used for determining cytokines and chemokines using rtPCR.

One lung from each mouse in the previous paragraph and from Example 7 are collected at the time of sacrifice and 16S sequencing is performed (see, e.g., Chen, J., et al. Genome Med 8: 43 (2016).). Samples are frozen and 16S rRNA sequencing is performed together for all samples. Phylotype profiles of the microbiome populations are generated using deep rDNA sequencing of the hypervariable V4 region of the 16S ribosomal RNA (rRNA) gene (see, e.g., Evaluation of 16S rDNA-Based Community Profiling for Human Microbiome Research. PloS one 7: e39315 (2012).). Microbial DNA is extracted using the MoBio PowerSoil Kit with a bead-beating step. A polymerase chain reaction (PCR) is performed using 50 ng cDNA and 0.3 μM barcoded primers with Kapa HiFi Hotstart Ready Mix. The data is analyzed as in Example 7 to confirm how treatment changes the microbial composition.

### Example 9

Data Interpretation Plan

Differences in lung compliance are determined by analysis using ANOVA with 7 mice in each group.

Analysis of microbiome: Samples are sequenced on one lane of MiSeq at the Mayo Genomics Facility using the MiSeq Reagent Kit v2 (500-cycles) (Illumina Inc.), generating 20M 2×250 reads. Pre-processed sequence files are processed by IM-TORNADO (Jeraldo, P., et al. PloS one 9: e114804, 2014.) removing poor quality and incomplete sequences. Sequences are then fed through the Ribosomal Database Project in order to obtain the taxa calls that are used in later correlation analysis. The bioinformatics pipeline allows operational taxonomic unit (OTU) assignments followed by microbial community analyses including sequence alignment, phylogenetic trees, phylogenetic and taxon-based analysis of diversity and network analysis as well as Unifrac analysis for clustering into OTUs, generating rarefaction curves and calculating the species richness estimation ACE (abundance-based coverage estimator) and Chaol (Chao 1 estimator) values. Coordinate analysis is based on the Bray-Curtis distances at the phylum-level which allows the testing of clustering of samples. Bray-Curtis distance matrices used for these analyses are calculated at various phylogenetic levels, including DNA sequence level, binning at phylum level, division level, genus level, or selective grouping at a combination of genus and species levels. The Bray-Curtis dissimilarity index is used to calculate the distance matrix for cluster analysis using the PHYLIP package. Bray-Curtis distance analysis, coordinate and statistical analyses used to cluster microbial profiles are performed as described (see, e.g., Chen, J., et al. Genome Med 8: 43, 2016.). Predictive profiling and other statistical analyses are performed in R-3.0.2 (R Development Core Teams) as described elsewhere (see, e.g., Chen, J., et al. Genome Med 8: 43, 2016.).

Statistical Power: 15 mice/strain are used to define changes in microbial profile (see, e.g., Chen, J., et al. Genome Med 8: 43, 2016.). Statistical power analysis of clustering is evaluated by two-group MANOVA method (see, e.g., Cohen, J. 1988. Chapter 10. In Statistical Power Analysis for the Behavioral Sciences, 2nd ed. Lawrence Erlbaum Assoc Inc. 567.). 15 mice per cohort provide significant results relating to the clustering of samples and allow for testing of the hypothesis that there is a characteristic change in the microbial profile of treated and untreated mice. Cytokine responses are analyzed by non-parametric student's t test. Two-sided p-values <0.05 are considered to be statistically significant.

### Example 10

Commensals are supplemented in oral cavity and changes in the lung microbiome in treated and untreated mice are measured. Oral supplementation of commensals leads to presence of the commensals in the gut. If no difference in CS-induced treatment is observed, mice are treated with antibiotic Vancomycin (which only impacts the gut) to confirm the impact of gut microbiota on CS-induced emphysema.

For gut microbiome testing, stool samples are collected before treatment begins and after treatment stops. Even when oral supplement changes gut microbiota only, differences in lung immune response after treatment can be confirmed given the gut-lung axis that exists.

### Example 11

Nasal application of microbes for lung-specific effect are confirmed as described for oral application of microbes.

### Example 12

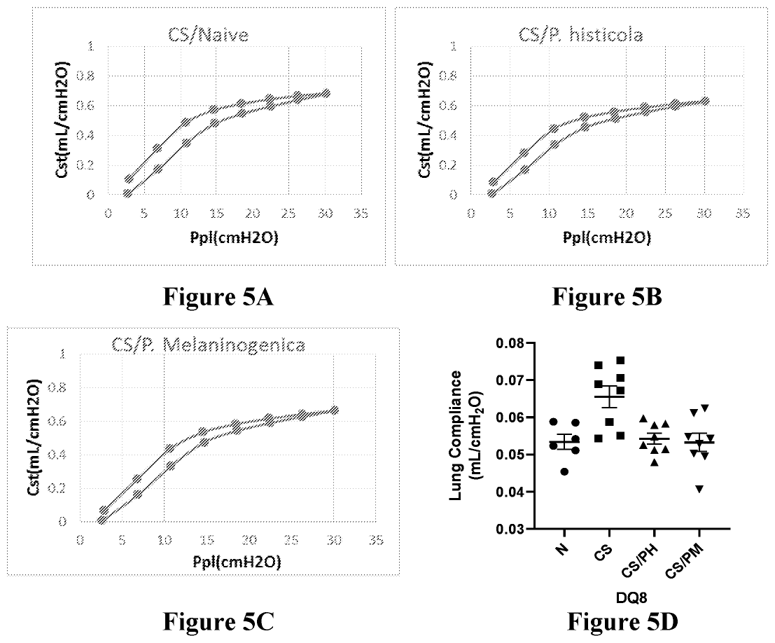

Naïve DQ8 mice were exposed to cigarette smoke (CS) or not (N) for 4 weeks. Two groups of mice exposed to CS for 2 weeks were treated with Prevotella histicola (PH) or Prevotella melaninogenica (PM) intranasally on alternate days. The data shows a significant loss of lung function with increased compliance indicative of emphysema or COPD for mice not treated with PH or PM (FIGS. 5A and 5D). N vs CS, P<0.008. After treatment with Prevotella histicola or Prevotella melaninogenica, mice showed compliance similar to that of naïve DQ8 mice exposed to air only (N) (FIGS. 5B-D). CS/PH vs CS, P<0.004, CS/PM vs CS, P<0.006. Analysis was done in using Prism software and unpaired T test.

Lung function was measured as static lung compliance (Cst) using a Flexivent system. Quantitative assessment of static lung compliance, volume and airway resistance were determined. Static lung compliance, airway and tissue resistance were measured under spontaneous ventilation. Baseline measurements of respiratory system resistance, compliance and elastance were done during a 2-s breath hold with a 2.5-Hz sinusoidal oscillation using the single-compartment model. Within 4 weeks of exposure to CS, mice had significant loss of lung function. Lung function mechanics is a terminal event, so mice after CS exposure for 2 weeks were treated intranasally for 2 weeks alternate days with live bacteria (10\hat{ }9 CFU/100 μL) Prevotella histicola or Prevotella melaninogenica suspended in 100 microliters of TSB (anaerobic) bacterial culture.

Cigarette Smoke exposure: Chronic CS exposure was performed in a smoking chamber (Teague enterprises, CA,) that enables exposure of mice to regulated concentrations of CS inhalation generated by 3R4F Kentucky research cigarettes (2 cigarettes every 10 minutes for 3 h/day on a daily basis for 5 days/week. In this system, mice are exposed to a mixture of mainstream and side-stream cigarette smoke and enables sufficient exposure that results in generation of murine blood nicotine levels analogous to levels attained in heavy human smokers (range of 45-181 ng/mL).

### Example 13

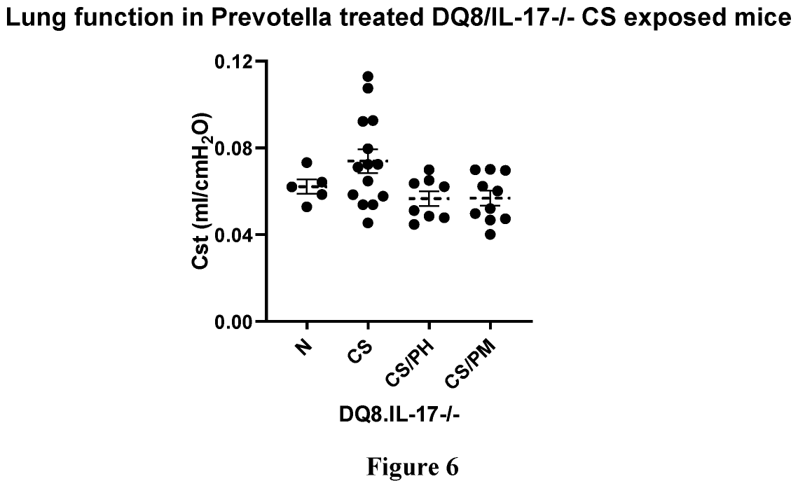

Naïve DQ8.IL-17^{-/-} mice were exposed to cigarette smoke (CS) or not (NS) for 4 weeks. Two groups of mice exposed to CS for 2 weeks were treated with Prevotella histicola (PH) or Prevotella melaninogenica (PM) intranasally on alternate days. The data in FIG. 6 shows that DQ8.IL-17^{-/-} naïve mice without exposure to CS have a significant loss of lung function as compared to naïve DQ8 mice (shown in FIG. 5A), P<0.048. After exposure to CS, loss of lung function in DQ8.IL-17^{-/-} mice was observed though it was not significant as compared to naïve mice suggesting IL-17 is essential for maintaining lung function. DQ8.IL-17^{-/-} mice exposed to CS were treated with Prevotella histicola or Prevotella melaninogenica as in Example 12. After treatment, mice showed compliance similar to that of naïve DQ8.IL-17^{-/-} mice exposed to air only (N). CS/PH vs CS, P<0.03, CS/PM vs CS, P<0.02. Treated DQ8. IL-17^{-/-} mice showed similar function as treated DQ8 mice suggesting Prevotella species could restore function that was lost due to IL-17 deficiency.

### Example 14

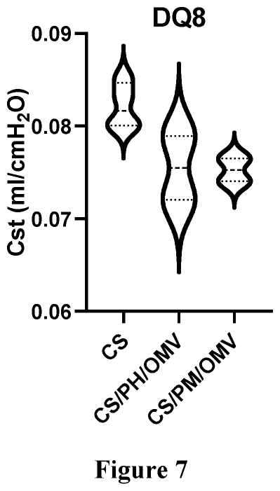

OMVs derived from Prevotella histicola and Prevotella melaninogenica were used to determine if these vesicles can improve lung function of mice with COPD. OMVs derived from P melaninogenica showed a significant improvement, CS vs CS/PM OMV, P<0.02 while those treated with OMVs derived from P. histicola showed variable responses with some mice showing restored lung function while another did not (FIG. 7). These data suggest that certain proteins in OMVs of Prevotella might be able to restore lung function of COPD.

OMVs were purified from in vitro broth culture of P. histicola and P. melaninogenica using ultracentrifugation method (Yang D., et al. Immunity. 2019; 50(3):692-706.e7. doi:10.1016/j.immuni.2019.02.001). Briefly, bacteria grown in anaerobic condition using Tryptic Soy Broth (TSB) were subjected into centrifugation for 15 min at 10,000 g (at 4° C.) to collect the bacteria-free supernatant. Collected supernatant were taken to ultra-centrifugation at 400,000 g for 1.5 hours (at 4° C.). After ultracentrifugation, supernatant was removed to get the pelleted OMVs. The OMV pellet was suspended in Phosphate Buffer Saline (PBS). Part of prepared OMVs suspension was plated on TSB-Blood Agar plate to check bacterial contamination.

Purified OMVs were biochemically characterized by quantifying total protein (Pierce BCA Protein Assay Kit—Thermo Fisher Scientific) to elucidate purity of OMVs and total LPS. DQ8 mice exposed to CS were treated with 100 mg (OMV lipopolysaccharide (LPS))/mouse intranasally.

### Example 15

Naïve DQ8 mice were exposed to cigarette smoke for 2 weeks. Mice were grouped in to 2 groups, one group was treated with intranasal application of P. histicola (PH) for 2 weeks and exposed to cigarette smoke while the other group was a control and only exposed to CS. * denotes P value for CS versus CS/PH. N=6 each group.

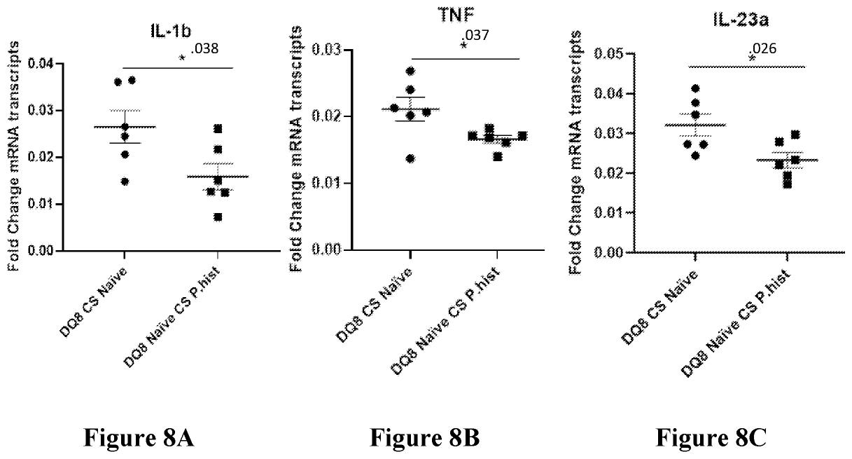

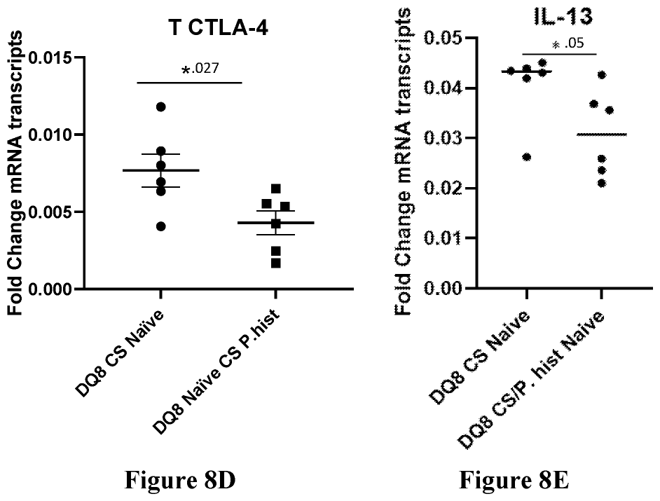

mRNA transcripts were measured by rtPCR using a commercially available kit (FIGS. 8A-E). Mice treated with P. histicola showed lower expression of proinflammatory cytokines. In addition, costimulatory CTLA-4 was reduced on T cells. IL-13 is known to induce activation and proliferation of B cells and production of antibodies. IL-23 is a TH17 cytokine and known to be involved in maintenance of TH17 response. Both IL-1b and IL-23 are increased in lungs of COPD patients and cause inflammation. TNF-a is a pleiotropic cytokine involved in cytotoxicity, angiogenesis, growth inhibition, inflammation and immunomodulation. TNF-a inhibitors have shown efficacy in severe COPD leading to less hospitalizations. These data suggest that mice with COPD receiving P. histicola treatment have less inflammation than those not treated.

### Example 16

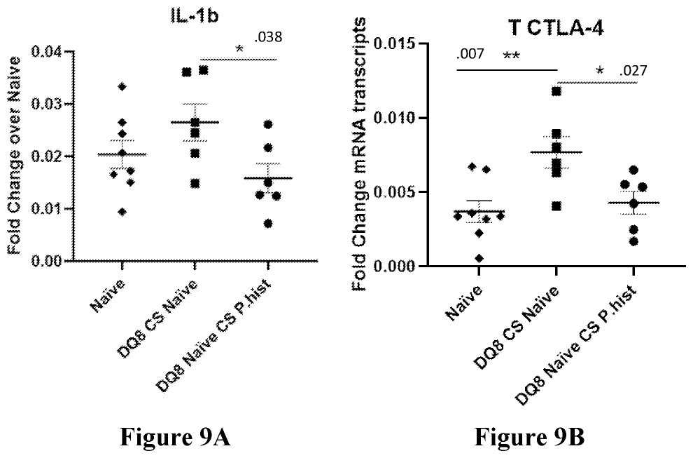

DQ8 naïve mice exposed to CS and treated with P. histicola showed levels of some cytokine transcripts reach the levels of naïve mice. IL-1B did not show any difference between naïve and P. histicola treated mice (FIG. 9A). Similarly, treated mice showed restored the CTLA expression levels similar to naïve mice (FIG. 9B).

Expression of all cytokines tested showed that no difference between the P. histicola treated and Naïve mice suggesting that P. histicola treatment normalizes the immune status in the lung of CS-exposed mice.

### Example 17

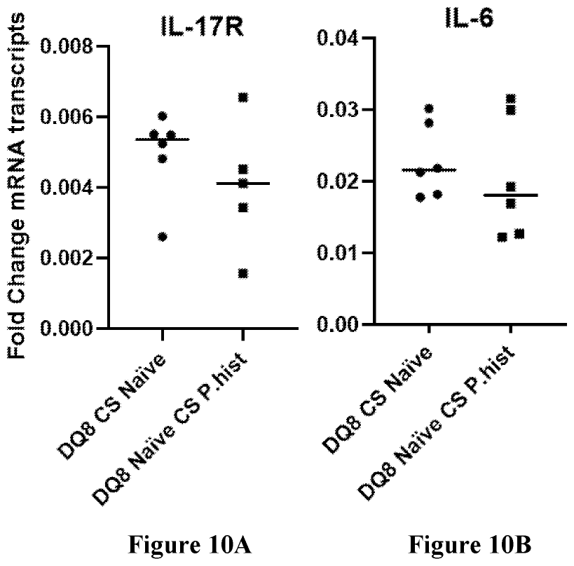

Treatment with P. histicola reduced levels of pro-inflammatory cytokines in CS-exposed naïve DQ8 mice as compared to CS naïve DQ8 mice (FIGS. 10A and 10B). However, differences were not significant suggesting that it reduces inflammation without causing immune suppression. Thus, such treatment does not result in being unable to fight infections.

### Example 18

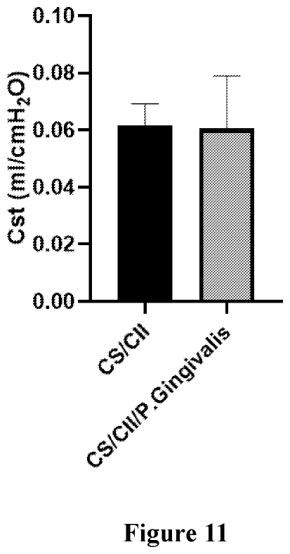

DQ8 mice were exposed to CS for 2 weeks and immunized with type II collagen (CII), N=4, for 4 weeks. Two mice were treated with Porphorymonas gingivalis intranasal for 2 weeks on alternate days while 2 mice did not receive any treatment. Lung compliance was analyzed after 4 weeks. No significant difference was observed (FIG. 11).

### Example 19

The experiment of Example 7 is repeated, using an additional group: Group 5—treated with Porphorymonas gingivalis. Lung function measurements are performed to confirm that P. histicola and P. melaninogenica suppress emphysema, while Porphorymonas gingivalis has no effect.

### Example 20

A human diagnosed with pneumonia or displaying one or more symptoms thereof (e.g., emphysema) is treated with a composition containing live P. histicola. The composition containing live P. histicola is administered orally to the human in a dose of about 1×10³ to about 1×10¹⁴ CFU once a day for 8 weeks. The human is monitored to confirm reduction of one or more symptoms of pneumonia.

### Example 21

A human diagnosed with pneumonia or displaying one or more symptoms thereof (e.g., emphysema) is treated with a composition containing live P. histicola. The composition containing live P. histicola is administered nasally to the human in a dose of about 1×10³ to about 1×10¹⁴ CFU once a day for 8 weeks. The human is monitored to confirm reduction of one or more symptoms of pneumonia.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating chronic obstructive pulmonary lung disease (COPD) in a mammal, wherein said method comprises administering a composition comprising live or killed Prevotella to the mammal.

2. The method of claim 1, wherein the composition comprises live Prevotella.

3. The method of claim 1, wherein the composition comprises killed Prevotella and no live Prevotella.

4. The method of claim 1, wherein administering comprises oral administration.

5. The method of claim 1, wherein administering comprises administration to the respiratory system.

6. The method of claim 1, wherein the composition comprises about 1×10³ to about 1×10¹⁴ CFU of Prevotella.

7. The method of claim 1, wherein the Prevotella comprises Prevotella histicola.

8-17. (canceled)

18. A method for treating a lung condition in a mammal, wherein said method comprises administering a composition comprising live or killed Prevotella melaninogenica to the mammal.

19. The method of claim 18, wherein the composition comprises live Prevotella melaninogenica.

20. The method of claim 18, wherein the composition comprises killed Prevotella melaninogenica and no live Prevotella melaninogenica.

21. The method of claim 18, wherein the composition comprises about 1×10³ to about 1×10¹⁴ CFU of Prevotella melaninogenica.

22. The method of claim 18, wherein the lung condition is selected from the group consisting of chronic obstructive pulmonary disease (COPD), asthma, pulmonary fibrosis, sarcoidosis, disease caused by infection, and combinations thereof.

23. The method of claim 22, wherein the disease caused by infection is selected from the group consisting of a viral infection, a bacterial infection, a fungal infection, a parasitic infection, and combinations thereof.

24. The method of claim 23, wherein the viral infection is selected from the group consisting of viral pneumonia, viral bronchitis, influenza, coronavirus infection, adenovirus infection, syncytial virus infection, rhinovirus infection, and combinations thereof.

25. A method for treating a lung condition in a mammal, wherein said method comprises administering a composition comprising vesicles of a Prevotella species.

26. The method of claim 25, wherein the Prevotella species comprises Prevotella histicola.

27. The method of claim 25, wherein the Prevotella species comprises Prevotella melaninogenica.

28. The method of claim 25, wherein the lung condition is selected from the group consisting of chronic obstructive pulmonary disease (COPD), asthma, pulmonary fibrosis, sarcoidosis, disease caused by infection, and combinations thereof.

29. The method of claim 28, wherein the disease caused by infection is selected from the group consisting of a viral infection, a bacterial infection, a fungal infection, a parasitic infection, and combinations thereof.

30. The method of claim 29, wherein the viral infection is selected from the group consisting of viral pneumonia, viral bronchitis, influenza, coronavirus infection, adenovirus infection, syncytial virus infection, rhinovirus infection, and combinations thereof.
